SEQUENCE LISTING (33 sequences)

SEQ ID NO: 1 - protein, 5 aa, Homo sapiens
ADARM

SEQ ID NO: 2 - protein, 5 aa, Homo sapiens
HSYQE

SEQ ID NO: 3 - protein, 6 aa, Homo sapiens
DHSYQE

SEQ ID NO: 4 - protein, 5 aa, Homo sapiens
RNVRF

SEQ ID NO: 5 - protein, 5 aa, Homo sapiens
VTLRI

SEQ ID NO: 6 - protein, 5 aa, Homo sapiens
IENLH

SEQ ID NO: 7 - protein, 7 aa, Homo sapiens
NLHRTFE

SEQ ID NO: 8 - protein, 5 aa, Homo sapiens
NLHRT

SEQ ID NO: 9 - protein, 5 aa, Homo sapiens
LHRTF

SEQ ID NO: 10 - protein, 5 aa, Homo sapiens
HRTFE

SEQ ID NO: 11 - protein, 5 aa, Homo sapiens
KGTQF

SEQ ID NO: 12 - protein, 8 aa, Homo sapiens
DNEVFGEA

SEQ ID NO: 13 - protein, 5 aa, Homo sapiens
DNEVF

SEQ ID NO: 14 - protein, 5 aa, Homo sapiens
NEVFG

SEQ ID NO: 15 - protein, 5 aa, Homo sapiens
EVFGE

SEQ ID NO: 16 - protein, 5 aa, Homo sapiens
VFGEA

SEQ ID NO: 17 - protein, 6 aa, Homo sapiens
QDTAVT

SEQ ID NO: 18 - protein, 5 aa, Homo sapiens
QDTAV

SEQ ID NO: 19 - protein, 5 aa, Homo sapiens
DTAVT

SEQ ID NO: 20 - protein, 5 aa, Homo sapiens
PKNAW

SEQ ID NO: 21 - protein, 6 aa, Homo sapiens
DNTFKD

SEQ ID NO: 22 - protein, 5 aa, Homo sapiens
DNTFK

SEQ ID NO: 23 - protein, 5 aa, Homo sapiens
NTFKD

SEQ ID NO: 24 - protein, 6 aa, Homo sapiens
LQTIQE

SEQ ID NO: 25 - protein, 5 aa, Homo sapiens
LQTIQ

SEQ ID NO: 26 - protein, 5 aa, Homo sapiens
QTIQE

SEQ ID NO: 27 - protein, 7 aa, Homo sapiens
KDSHHPA

SEQ ID NO: 28 - protein, 5 aa, Homo sapiens
HGRTQ

SEQ ID NO: 29 - protein, 8 aa, Homo sapiens
YKDSHHPA

SEQ ID NO: 30 - protein, 5 aa, Homo sapiens
YKDSH

SEQ ID NO: 31 - protein, 5 aa, Homo sapiens
KDSHH

SEQ ID NO: 32 - protein, 5 aa, Homo sapiens
DSHHP

SEQ ID NO: 33 - protein, 5 aa, Homo sapiens
SHHPA

(19) United States
(12) Patent Application Publication
(10) Pub. No.: US 2014/0134197 A1
(43) Pub. Date: May 15, 2014

(54) Mimotopic Peptides for the Diagnosis and Treatment of Multiple sclerosis

(71) Applicant: Emanuel Calenoff, Dallas, TX (US)

(72) Inventor: Emanuel Calenoff, Dallas, TX (US)

(73) Assignee: Emanuel Calenoff, Dallas, TX (US)

(21) Appl. No.: 13/423,119

(22) Filed: Mar. 16, 2012

Related U.S. Application Data
(60) Provisional application No. 61/453,747, filed on Mar. 17, 2011.

(51) Int. Cl. C07K 2/00; G01N 33/68

(52) U.S. Cl. CPC C07K 2/00; G01N 33/6896
USPC 424/185.1; 530/300; 436/501

(57) ABSTRACT

The present invention provides peptides and methods for diagnosing and treating multiple sclerosis and wherein the method can also be applicable to the diagnosis and treatment of other immune disorders.

### BACKGROUND

Multiple sclerosis (MS) is an autoimmune inflammatory disease in which fatty myelin sheaths surrounding the axons of the brain and spinal cord are damaged, leading to demyelination, scarring and a broad spectrum of signs and symptoms. MS is often a debilitating illness that can cause premature death.

The present invention relates to the role of IgE antibodies that bind to myelin proteins and their initiation and sustenance of the MS autoimmune process.

Until now, the vast majority of immunoassays and diagnostic procedures for detecting MS were not focused upon the role of dimeric IgE binding to myelin proteins that result in focal mast cell degranulation and MS lesion causation. Previous techniques that have measured IgE have not disclosed the appropriate peptide combinations for such use.

The present invention uses mimotopic peptides for immunoassays that provide greater than 95% sensitivity in MS detection, as opposed to previous methods that provide only about 60% sensitivity. A new and unique approach is the method of estimating the distance between two or more IgE-bound epitopes. That method entails counting the amino acids between epitopes and multiplying the intervening amino acid number times 10.6 Ångströms per amino acid. Alternatively, one could summate the specific diameters of the individual amino acids constituting the specific protein. If the resulting epitope interval is 40 to 100 Ångströms, then mast cell degranulation with focal release of tissue damaging enzymes and/or other deleterious substances is likely to be taking place.

Once identified the deleterious process can be abrogated or reduced by in-vivo administration of similarly structured peptides to neutralize the antibody-mediated process.

Therapeutic peptide selection can be limited to the use of one of two dimer-point peptides while still attaining a high measure of therapeutic efficacy and multiple dimer site coverage.

### SUMMARY

The invention provides isolated peptides homologous to individual myelin protein epitopes, wherein each peptide has a net hydrophilicity index value of about −2.5 to 6.3 and can be used as a component of disease-specific diagnostic tests and matching therapeutic compositions.

A mimotopic peptide antigen-based immunoassay used to measure in-vivo IgE excess is described wherein the ratio of epitope-specific IgE relative to its matching, competing non-IgE antibodies is determined. The positive dimeric presence of specific IgE excess is an indication of disease presence.

Also described is therapeutic method in which the matching mimotopic peptides are used to neutralize epitope-specific autoantibodies.

### BRIEF DESCRIPTION OF THE DRAWINGS

FIG. 1a is a schematic illustration of myelin proteolipid protein Isoform 1. Depicted are: (1) amino acid sequence portions that are net hydrophilic (boxed) and located on the myelin protein (oligodendrocyte) surface; (2) portions that are net hydrophobic and project inwardly within the myelin glycolipid layer (unboxed); and (c) portions that are hydrophilic and are intracellular (also boxed but at figure bottom).

FIG. 1b schematically depicts Proteolipid protein Isoform 1 (PLP 1) as an in-folded Hopp and Woods XY plot with eleven vertical datacolumns. The left-most column depicts the amino acid sequence number of the outer surface portions of the protein chain. The second column from the left lists the surface portions' corresponding amino acids. The sixth, left-most, column displays the hydrophilic index (HI) of each depicted amino acid as if analyzed alone. The tenth column from the left depicts the sum-of-seven, continuous amino acids, hydrophilic index value of each amino acid which is derived by adding to its hydrophilic index (HI) the indices of the 3 amino acids that precede it plus the indices of the three amino acids that follow it. Areas that are net hydrophilic (boxed amino acids) are apt to be on the protein surface while those that are net hydrophobic (not boxed) would be on the protein edge or located within the protein center. The protein surface can either be extracellular or intracellular. In multiple sclerosis, the oligodendrocyte extracellular PLP humoral epitope ADARM (SEQ ID NO: 5) is significantly immunogenic. Its humoral epitopic footprint encompasses its unique pentameric sequence plus a normally located amino acid on either end.

FIG. 2 displays is the measured distance between two IgE autoantibodies if each was to bind a potential epitopic dimer site (VTLRI (SEQ ID NO: 5) and HSYQE (SEQ ID NO: 2)) with each site incorporating five, uniquely sequenced, contiguous amino acids flanked on either end by a non-reactive, normally present amino acid thus making a 7 amino acid, antibody-binding footprint. Each intervening amino acid between epitopes is estimated to be 10.6 Ångströms in width. When the inter-footprint dimer distance analysis is performed, the potential dimer between VTLRI (SEQ ID NO: 5) and HSYQE (SEQ ID NO: 2) is adequate for mast cell degranulation because there are 12 intervening amino acids between the two epitopes, and this is equivalent to a distance of 95 Ångströms, which is within the mandated upper limit of 100 Ångströms.

FIG. 3 illustrates a potentially functional dimer site with an interval distance of 56 Ångströms between the epitopes RNVRF (SEQ ID NO: 4) and HSYQE (SEQ ID NO: 3).

FIG. 4 illustrates a potentially functional dimer site with an interval distance of 95 Ångströms between the subsurface MOG epitopes IENLH (SEQ ID NO: 6) and KTGQF (SEQ ID NO: 11). The epitopes' complexing with specific IgE antibodies likely hinges upon disruption of the overhanging oligodendrocyte membrane surface and inflow of cerebrospinal fluid that contains myelin epitope-specific autoantibodies.

FIG. 5 Illustrates are three potentially functional dimer sites with interval distances of 80, 71, and 64 Ångströms between the intracellular MOG epitopic dimer sites NLHRT (SEQ ID NO: 8) and KTGQF (SEQ ID NO: 11), LHRTF (SEQ ID NO: 9) and KTGQF (SEQ ID NO: 11), and HRTFE (SEQ ID NO: 10) and KTGQF (SEQ ID NO: 11). Dimeric IgE complexing hinges upon disruption of the overhanging oligodendrocyte membrane surface and facilitated intracellular autoantibody inflow. For serum antibody immunoassay purposes, the longer, inclusive peptide NLHRTFE (SEQ ID NO: 7) can be used together with KTGQF (SEQ ID NO: 11) as both peptides are sufficiently hydrophilic when coupled with the peptide-solubilizing 8-Fmoc-amino-3,6-dioxa-octanoic acid₂ (amino-ADOOA-ADOOA) linker.

FIG. 6 illustrates seven structurally unique epitopes located on the surface of myelin basic protein (MBP) Isoform 1 should it become exposed to autoantibody binding. The dimer group 1 encompasses the dimer epitope pairs DNEVF (SEQ ID NO: 13) and QDTAV (SEQ ID NO: 18), NEVFG (SEQ ID NO: 14) and QDTAV (SEQ ID NO: 18), EVFGE (SEQ ID NO: 15) and QDTAV (SEQ ID NO: 18), and VFGEA (SEQ ID NO: 16) and QDTAV (SEQ ID NO: 18). The dimer group 2 encompasses the dimer epitope pairs DNEVF (SEQ ID NO: 13) and DTAVT (SEQ ID NO: 19), NEVFG (SEQ ID NO: 14) and DTAVT (SEQ ID NO: 19), EVFGE (SEQ ID NO: 15) and DTAVT (SEQ ID NO: 19), and VFGEA (SEQ ID NO: 16) and DTAVT (SEQ ID NO: 19). The dimer group 3 encompasses the dimer epitope pairs QDTAV (SEQ ID NO: 18) and PKNAW (SEQ ID NO: 20) and DTAVT (SEQ ID NO: 19) and PKNAW (SEQ ID NO: 20). Dimer group 1 displays epitope intervals that are 95, 87, 80, and 72 Ångströms. Dimer group 2 displays epitope intervals that are 88, 80, 72, and 64 Ångströms. Dimer group 3 displays epitope intervals that are 48 and 40 Ångströms. Dimeric IgE complexing hinges upon disruption of the overhanging Myelin (oligodendrocyte membrane) surface and facilitated intracellular autoantibody inflow. For screening serum autoantibody, immunoassay purposes, the longer, inclusive peptide DNEVFGEA (SEQ ID NO:12) can be used together with QDTAVT (SEQ ID NO: 17) and QDTAVT (SEQ ID NO:17) used together with PKNAW (SEQ ID NO: 20) as all three peptides are sufficiently hydrophilic when coupled with the peptide-solubilizing amino-ADOOA-ADOOA linker. Individual confirming tests would employ the epitope-matching, pentameric constituents of the larger peptides.

FIG. 7 displays a second set of potentially functional intracellular dimer sites found on the inwardly projecting surface of the protein MBP Isoform 1. The inclusive epitope pairs are: DNTFK (SEQ ID NO: 22) and LQTIQ (SEQ ID NO: 25), DNTFK (SEQ ID NO: 22) and QTIQE (SEQ ID NO: 26), NTFKD (SEQ ID NO: 23) and LQTIQ (SEQ ID NO: 25) plus NTFKD (SEQ ID NO: 23) and QTIQE (SEQ ID NO: 26) with respective interval distances of 40 and 48 Ångströms. Dimeric IgE complexing hinges upon disruption of the overhanging myelin (oligodendrocyte membrane) surface and facilitated intracellular autoantibody inflow. For screening serum antibody immunoassay purposes, the longer, inclusive peptide DNTFKD can be used together with LQTIQE (SEQ ID NO: 24) as both peptides are sufficiently hydrophilic when coupled with the peptide-solubilizing, amino-ADOOA-ADOOA linker. Individual confirming tests would employ the pentameric equivalents of the larger peptides.

FIG. 8 displays third set of potentially functional intracellular dimer sites on MBP Isoform 1, encompassing the epitope pairs KDSHH (SEQ ID NO: 31) and HGRTQ (SEQ ID NO: 28), DSHHP (SEQ ID NO: 32) and HGRTQ (SEQ ID NO: 28), plus SHHPA (SEQ ID NO: 33) and HGRTQ (SEQ ID NO: 28). The respective, displayed epitopic intervals are 42, 64, 56 Ångströms. The dimer epitopes' complexing with specific IgE antibodies hinges upon disruption of the overhanging myelin (oligodendrocyte) surface and epitope-specific antibody inflow. For serum antibody immunoassay purposes, the longer, inclusive peptide KDSHHPA (SEQ ID NO: 27) can be used together with HGRTQ (SEQ ID NO: 28) as both solubilize readily with the amino-ADOOA-ADOOA peptide linker. Individual confirming tests would employ the pentameric equivalents of the larger peptides.

FIG. 9 illustrates a potentially functional dimer sites on MBP Isoform 2 whose conditions that match the dimer sets on MBP Isoform 1 displayed in FIG. 6

FIG. 10 depicts potentially functional dimer sites and conditions on MBP Isoform 2 that match a dimer set on MBP Isoform 1 as displayed in FIG. 7.

FIG. 11 illustrates a set of potentially functional intracellular dimer sites on MBP Isoform 3 encompassing the epitope pairs YKDSH (SEQ ID NO: 30) and HGRTQ (SEQ ID NO: 28), KDSHH (SEQ ID NO: 31) and HGRTQ (SEQ ID NO: 28), DSHHP (SEQ ID NO: 32) and HGRTQ (SEQ ID NO: 28), and SHHPA (SEQ ID NO: 33) and HGRTQ (SEQ ID NO: 28). The respectively displayed epitopic intervals are 82, 74, 67, and 60 Ångströms. The dimer epitopes' complexing with specific IgE antibodies hinges upon disruption of the overhanging myelin (oligodendrocyte) surface and specific antibody inflow. For serum antibody immunoassay purposes, the longer, inclusive peptide YKDSHHPA (SEQ ID NO: 29) can be used together with HGRTQ (SEQ ID NO: 28) as both solubilize readily with the amino-ADOOA-ADOOA peptide linker. Individual confirming tests would employ the pentameric equivalents of the larger peptide.

FIG. 12 depicts example MS test results for ten female control serum samples, ages 20-66. Tested samples were from Caucasian and African-American donors who did not have multiple sclerosis. Specific IgE/(kappa+lambda)-positive results are confined to single, non-dimer participating epitopes (m.s.=myelin, oligodendrocyte cell surface protein portion; i.c.=myelin, oligodendrocyte intracellular protein's or protein portion's surface).

FIG. 13 depicts example MS test results for ten male control serum samples, ages 24-66. Tested samples are from Caucasian and African-American donors who did not have multiple sclerosis. Specific IgE/(kappa+lambda)-positive results are confined to single, non-dimer participating epitopes.

FIG. 14 depicts example MS test results for serum samples obtained from multiple sclerosis patients (4 Caucasians and 1 African American) who had not yet received pharmacotherapy. Individual epitope-positive results are block-highlighted. To be dimer test-positive, MS patients had to be ADARM (SEQ ID NO: 1) IgE/(kappa+lambda)-positive and/or IgE/(kappa+lambda)-positive for the dimer pairs HSYQE (SEQ ID NO: 2) and VTLRI (SEQ ID NO: 5), HSYQE (SEQ ID NO: 2) and RNVRF (SEQ ID NO: 4), IENLH (SEQ ID NO: 6) and KTGQF (SEQ ID NO: 11), NLHRT (SEQ ID NO: 8) and KTGQF (SEQ ID NO: 11), LHRTF (SEQ ID NO: 9) and KTGQF (SEQ ID NO: 11), and/or HRTFE (SEQ ID NO: 10) and KTGQF (SEQ ID NO: 11).

FIG. 15 depicts example MS test results for serum samples obtained from multiple sclerosis patients only treated with interferon or Copaxone. Individual epitope-positive results (all against the PLP epitope ADARM (SEQ ID NO: 1)) are block-highlighted. Being test-positive to PLP indicates dimer-positive presence because of the PLP monomers' high myelin surface prevalence and adequate intermolecular monomer-to-monomer separation (65-71 Ångströms).

FIG. 16 depicts MS test results for serum samples obtained from multiple sclerosis patients treated with interferon plus psychotropic pharmaceuticals and/or other potentially immunosuppressive a gents. Individual dimer-positive results (just one) are block-highlighted. The immunosuppressive (or immunoassay altering) substances are identified by vertically-placed numbers at the bottom of columnar, individual patient test results and referenced in literary citations provided in Tables 3a-e that are listed at the end of the application.

FIG. 17 depicts MS test results for serum samples obtained from multiple sclerosis patients only treated with psychotropic pharmaceuticals or other potentially immunosuppressive agents. Individual dimer-positive results (4 ADARM-positives) are block-highlighted. The immunosuppressive (or immunoassay altering) substances are identified by vertically-placed numbers at the bottom of columnar, individual patient test results and referenced in literary citations provided in Tables 3a-e that are listed at the end of the application.

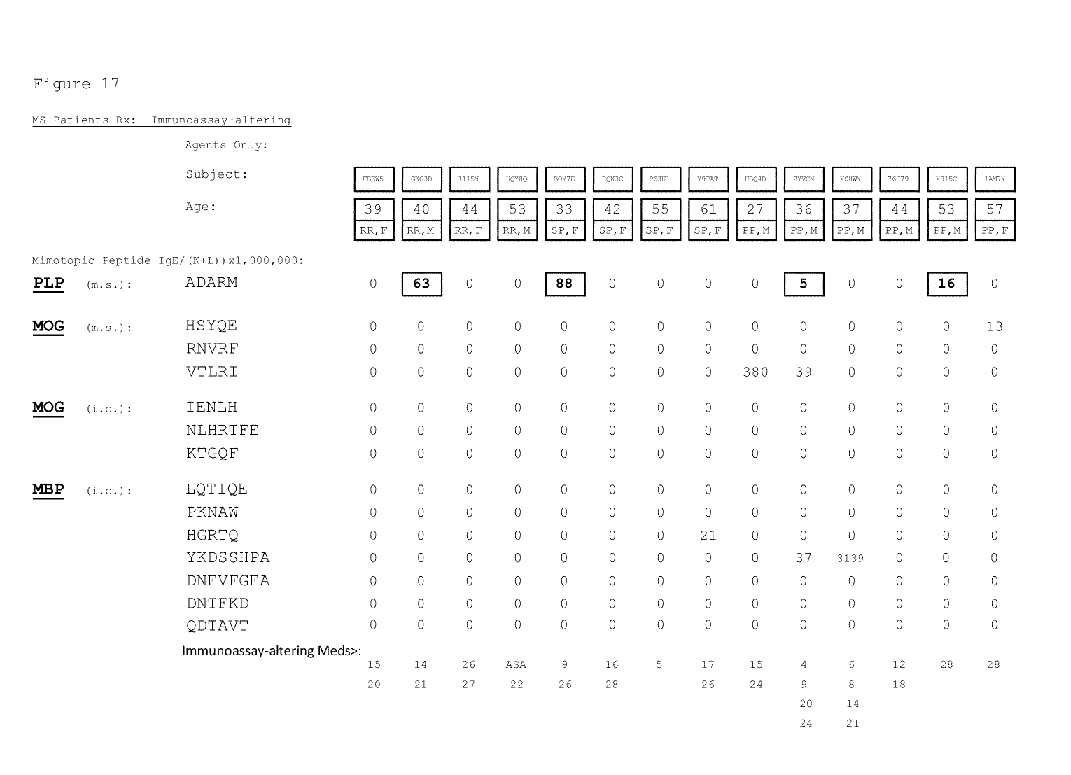

FIG. 18 depicts MS test results for serum samples obtained from multiple sclerosis patients treated with Copaxone plus psychotropic pharmaceuticals or other potentially immunosuppressive agents. Individual epitope-positive results (just one tested individual) are block-highlighted. The immunosuppressive (or immunoassay altering) substances are identified by vertically-placed numbers at the bottom of columnar, individual patient test results and referenced in literary citations provided in Tables 3a-e that are listed at the end of the document.

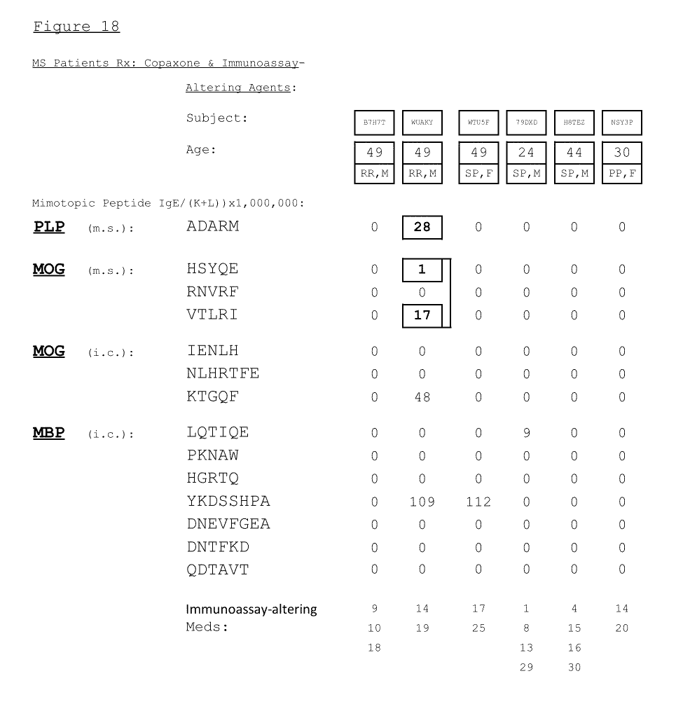

Table 1 illustrates the method employed in estimating the average diameter, in Ångströms, of the twenty standard amino acids. The method entails: (1) Estimating the nanometers diameter of each non-alanine amino acid relative to the known diameter of alanine, 0.69 nanometer, using the formula amino acid molar mass/alanine molar mass×0.69 nanometer; (2) multiplying each estimated amino acid diameter times 10 in order to convert individual amino acid diameter from nanometers to Ångströms; and (3) summing the Ångströms diameters and dividing by 20 to yield an average, estimated amino acid diameter of 10.6 Ångströms.

Table 2 lists the concentration of individual mimotopic peptide constructs used in application to individual MS assay, microplate test wells alongside each construct's peptide amino acid sequence.

Table 3a thru 3e list the psychotropic pharmaceuticals and other therapeutic agents shown to be immunosuppressive (left column) alongside their specific suppressive effects (middle column) and describing literary citations (right column). Citations are listed in the numbered patent References section.

Table 4a lists structurally unique, mimotopic, peptides serving as diagnostic and therapeutic antigens. Respective peptide hydrophilic indices (HI) are displayed in columns 4 and 6. Peptides used for initial diagnosis can be of maximum, unique length (column 3) or can be fractionated into pentameric, single epitope equivalents (column 5). Each test peptide is synthesized with an 8-amino-3,6-dioxaoctanoic acid₂) linker. The amide group is used for covalent Coupling to microplate wells. The 3,6-dioxaoctanoic acid₂ construct, being very hydrophilic, solubilizes most al peptides, especially those that are relatively hydrophobic. The listed myelin, dimer-cornerstone peptide homologues (bold-highlighted) for MS therapy are useful because of their in-vivo: (a) net hydrophilicity for adequate solubility; (b) relatively small size for intravascular permeation; and (c) ability to simultaneously abrogate formation of about 20 pathological myelin dimers by administering just 6 peptides (bold highlighted in column 5) in lieu of needing to employ up to 25 individual therapeutic pentamers and also having to confront different solubility and molecular aggregation issues.

Table 4b lists the diagnostic construct derivatives of the pentameric peptides listed in Table 4a wherein each has attached the solubilizing linker Fmoc-8-Amino-3,6-Dioxaoctanoic Acid-Fmoc-8-Amino-3,6-Dioxaoctanoic Acid₂ (ADOOA-ADOOA). The linker provides both increased hydrophilicity for enhanced solubility and serves as a point of attachment onto microtiter test plate wells.

Table 4b illustrates the single-epitope, mimotopic peptides depicted in Table 4a divided into myelin outer surface (first separated group) and two myelin subsurface groups (second and third separations). Also depicted is a structural representation of each of the 25 peptide constructs coupled to microtiter test wells, an Fmoc-8-Amino-3,6-Dioxaoctanoic Acid₂ linker attached to a mimotopic peptide. The relative molar mass of each mimotopic peptide construct is listed in column 9. The molar mass of each corresponding mimotopic peptide, alone, is depicted in column 11. The hydrophilic index of each peptide construct was greater than 3 and that of each construct's corresponding pentameric peptide shown in column 12.

Table 5 depicts the average adult male serum IgA, IgG, and IgM antibody levels displayed along with the maximum possible serum-specific IgE level. Exhibited on the bottom is the estimated in-vivo half-life of each antibody isotype. Isotype-specific literature citations are numbered and provided in the References section.

Table 6 is an estimation of average molar quantity of individual antibody isotypes found in total serum volume of an average adult male as related to antigen-binding sites. Column (b) lists gram per milliliter of each isotype; column (c) lists individual quantities in mole/mL serum; column section (d) lists the mole/mL serum times the binding site valence number of each isotype to yield valence mole; and column section (e) depicts the valence mole of each antibody isotype of per 2,750 mL serum.

Table 7 depicts a beginning estimation of individual epitope-specific antibodies (in moles) specific for a single humoral epitope to be found in average adult male serum volume (2,750 mL) if the number of possible, discernible epitopes is estimated to be 1,000,000. The estimated mole value per humoral epitope would then be 0.00000000060164.

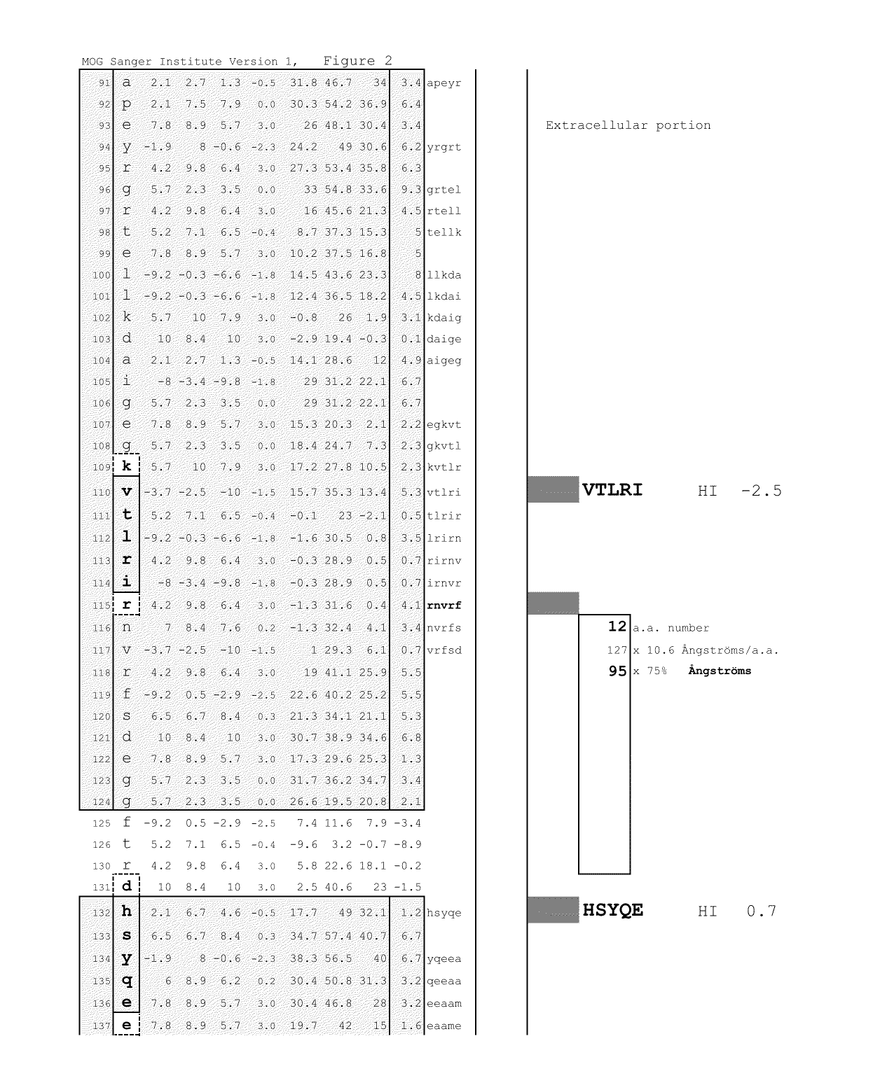

Table 8 is an estimation of the quantity of the ADARM (SEQ ID NO: 1), HSYQE (SEQ ID NO: 2), KTGQF (SEQ ID NO: 11) peptide mixture needed per day to block the dimer cornerstone epitopes on the outer and immediate subsurface of myelin. The potential MOG dimers are depicted on FIGS. 2-5. The epitope ADARM (mimicked by (SEQ ID NO: 1)) is located on individually spaced PLP monomers on lipid rafts and is described and illustrated in Reference 59.

Table 9 is an estimation of the maximum quantity of the 3 peptide mix needed per day to block the dimer cornerstone epitopes on the varied isoforms of myelin basic protein (MBP) located within the oligodendrocyte, intracellular portion of myelin if it were to be exposed. The potential MBP cornerstone epitope mimicking peptides are DNTFKD (SEQ ID NO: 21), HGRTQ (SEQ ID NO: 28), and QDTAVT (SEQ ID NO: 17).

Table 10 depicts the quantitative kinetics of an assumed 10 minute intravascular half-life, due to renal clearance and enzymatic degradation, of the PLP and MOG-derived peptides ADARM (SEQ ID NO: 1), DHSYQE (SEQ ID NO: 3) and KTGQF (SEQ ID NO: 11). The table displays the dilution range in which sufficient intravascular quantities of the 3 peptides would be available daily so as to abrogate corresponding cornerstone, epitope-specific IgE autoantibody binding and thus halt pathologic mast cell degranulation. The 10 minute half-life estimate was based on the work of Esposito (66). The derived table indicates that a weekly 40 mg (40,000 μg) therapeutic peptide injection provides a sufficient medicinal bolus to yield an adequate daily neutralizing dose of 1.46 μg. To be certain of therapeutic efficacy, epitope-specific serum IgE/(kappa+lambda) values are determined at reasonable time intervals following onset of therapy to titer the effect of the subcutaneously administered mimotopic peptides. The efficacy-targeted IgE/(kappa+lambda) goal is zero or almost zero. Successful neutralization is depicted in Table 12b.

Table 11 depicts an analysis similar to Table 10 is shown for myelin basic protein (MBP) cornerstone peptides mixture DNTFKD, HGRTQ, and QDTAVT. The in-vivo daily peptide dose requirement would be about 21.8 μg. Estimated weekly S.Q. injection would be about 52.6 mg.

Table 12a depicts raw test data corresponding to epitope-specific serum antibodies detected against the anterior myelin surface epitopes ADARM (exemplified by peptide (SEQ ID NO: 1)) and HSYQE (exemplified by peptide (SEQ ID NO: 2)). Columns 2 and 4 depict specific IgE chemiluminescence data points of a tested pretreatment serum sample from a sixty year-old secondary progressive MS male patient who had not received interferon, Copaxone, or chronic steroids. Columns 3 and 5 depict the post-eight week, treatment commencement data equivalents from the same patient, and the column 6 data points represent the raw IgE background data from both point sets. Columns 7 and 9 depict specific (kappa+lambda) chemiluminescence data points of the pretreatment tested serum sample and columns 8 and 10 depict the post-eight week, treatment commencement data equivalents. Column 11 data points are the raw (kappa+lambda) background data points. The highest and lowest point values in each column were eliminated, remaining values averaged, and standard deviation (SD) derived for the six remaining points in each column. Each respective column value was deemed to be its point average plus two standard deviations.

Table 12b depicts the MS activity factor (MAF) of each data point set derived by: (a) subtracting background point values from respective, corresponding peptide-well point values (b) multiplying each (kappa+lambda) value by 25,000 to adjust for serum dilution; (c) dividing each IgE point value by its corresponding adjusted (kappa+lambda) value; and (d) multiplying by 1,000,000 in order to attain whole number quotient values wherever possible. Dimeric, positive MAF values indicate the presence of myelin-dimer-generated pathology fostered by mast cell degranulation. Negative single-point MAF values are an indication of cessation or absence of MS autoimmune pathology. If the immunoassay is being used to monitor successful or failed specific IgE eradication, the testing process can be repeated periodically on an as need basis to make sure that the MAF values remain negative. The assay can also be used as an initial MS screening test. The eight-week follow-up of the MAF analysis of this tested patient shows post treatment initiation ADARM and HSYQE-negative results as compared to the MAF-positive pre-treatment results thus indicating probable therapeutic efficacy.

### DETAILED DESCRIPTION

### Detailed Description of the Invention

Multiple sclerosis (MS) is an autoimmune disease caused by a humoral pathological process that comprises interplay of damaging myelin epitope-specific IgE antibodies and competing, protective non-IgE antibodies. The non-IgE antibodies are specific IgA, IgG, and/or IgM. The cross-competing antibodies variably target 25 distinctive myelin binding sites, the epitopes. The amino acid sequences of the epitopes are: ADARM (SEQ ID NO: 1), HSYQE (SEQ ID NO: 2), RNVRF (SEQ ID NO: 4), VTLRI (SEQ ID NO: 5), IENLH (SEQ ID NO: 6), NLHRT (SEQ ID NO: 8), LHRTF (SEQ ID NO: 9), HRTFE (SEQ ID NO: 10), KGQF (SEQ ID NO: 11), DNEVF (SEQ ID NO: 13), NEVFG (SEQ ID NO: 14), EVFGE (SEQ ID NO: 15), VFGEA (SEQ ID NO: 16), QDTAV (SEQ ID NO: 18), DTAVT (SEQ ID NO: 19), PKNAW (SEQ ID NO: 20), DNTFK (SEQ ID NO: 22), NTFKD (SEQ ID NO: 23), LQTIQ (SEQ ID NO: 25), QTIQE (SEQ ID NO: 26), HGRTQ (SEQ ID NO: 28), YKDSH (SEQ ID NO: 30), KDSHH (SEQ ID NO: 31), DSHHP (SEQ ID NO: 32), and SHHPA (SEQ ID NO: 33).

When complexed with myelin in relative excess and in dimeric form, IgE antibodies are functionally bound by circulating mast cells causing the mast cells to degranulate and focally release proteolytic enzymes and other factors. The released enzymes and factors cause neuronal damage or destruction.

The invention provides isolated peptides that are individually homologous to myelin protein epitopes, wherein the peptides have net hydrophilicity values of about −2.5 to 6.7.

The peptides of the invention individually comprise 5 amino acids and are mimotopic, defined herein as structurally mimicking humoral epitopes on the surface of proteins or other molecules. The molecular sections upon which the epitopes are located can be extracellular or intracellular.

The peptides are not limited to but may be selected from or homologous in total or in part to any one of the following amino acid sequences: AAMEL, ADARM (SEQ ID NO: 1), HSYQE (SEQ ID NO: 2), DHSYQE (SEQ ID NO: 3), QAPEY, RNVRF (SEQ ID NO: 4), VTLRI (SEQ ID NO: 5), IENLH (SEQ ID NO: 6), NLHRTFE (SEQ ID NO: 7), KTGQF (SEQ ID NO: 11), DNEVFGEA (SEQ ID NO: 12), QDTAVT (SEQ ID NO: 17), PKNAW (SEQ ID NO: 20), DNTFKD (SEQ ID NO: 21), LQTIQE (SEQ ID NO: 24), YKDSHHPA (SEQ ID NO: 29), and HGRTQ (SEQ ID NO: 28).

Homologous is defined in this specification as being 100% identical to a corresponding sequence.

The invention also provides a composition comprising dimeric peptides, each of which is homologous to a myelin protein epitope, wherein a first epitope is located approximately 40-100 Angstroms from a second epitope. The epitope pairs can be located on protein sections found on the outer surface of myelin or within myelin layers wherein the relevant protein sections become exposed when an oligodendrocyte-contributed outer myelin surface is disrupted.

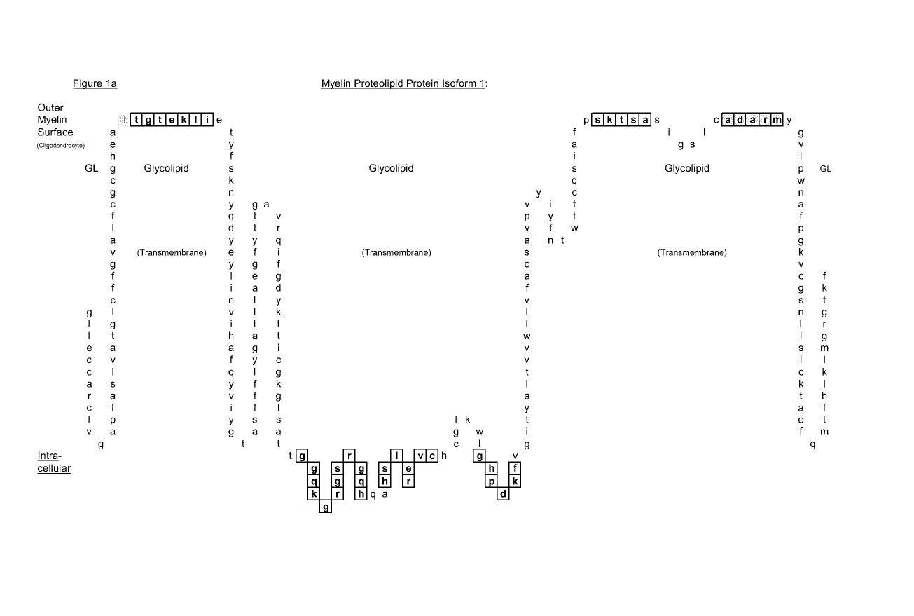

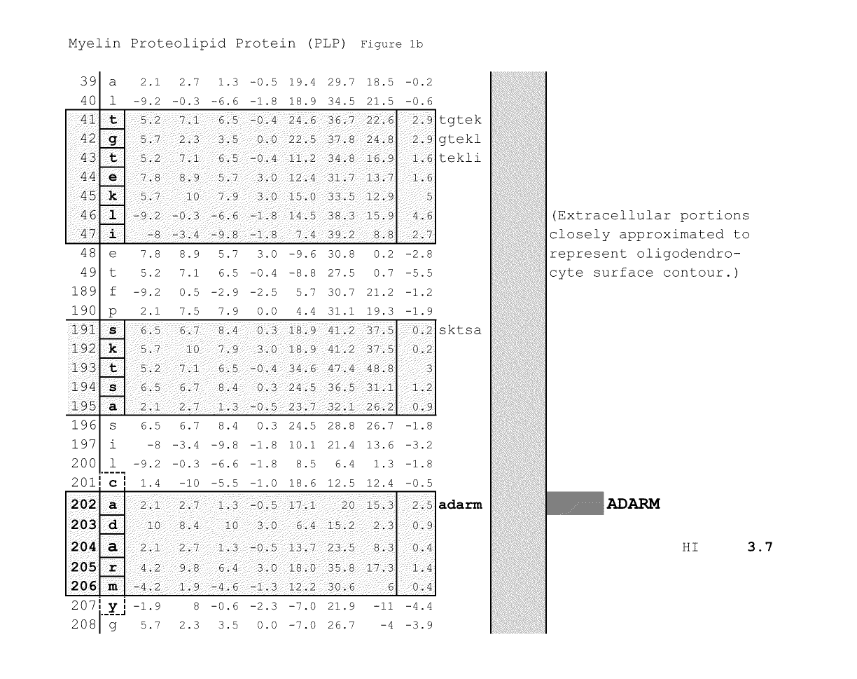

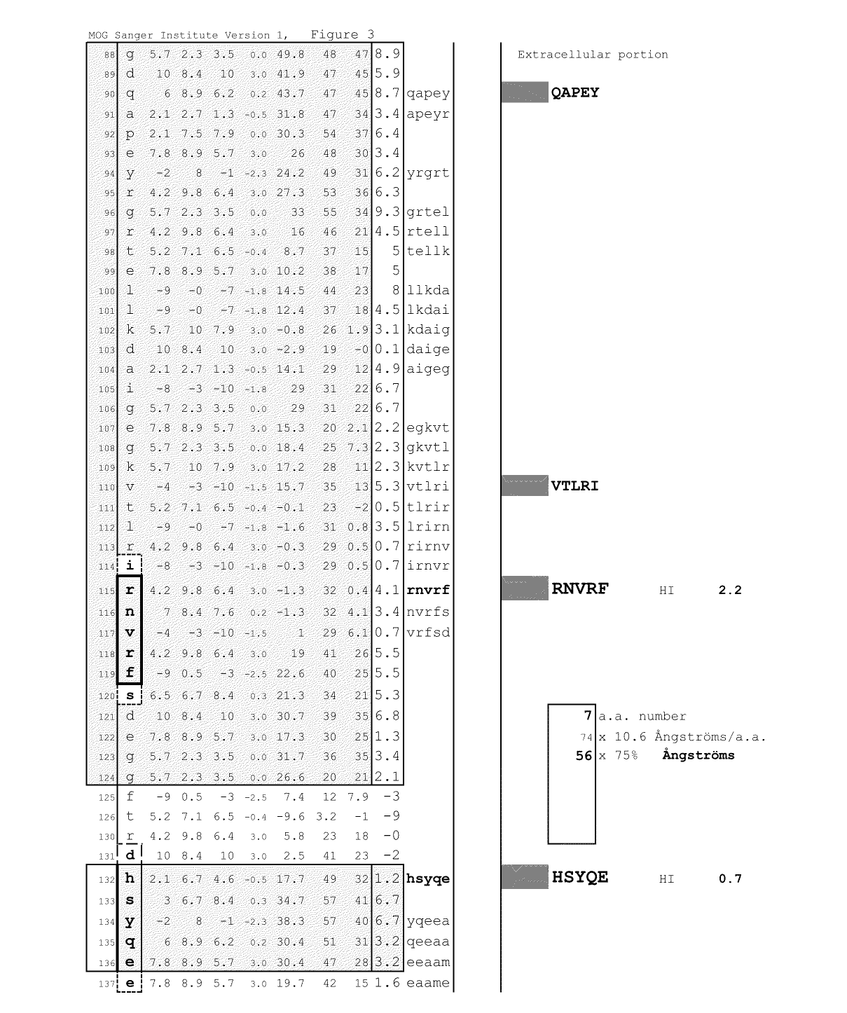

The outer surface epitope pairs comprise one univalent ADARM amino acid sequence, mimicked by (SEQ ID NO: 1), and a second, appropriately spaced, univalent ADARM sequence, both located on proteolipid protein (PLP) molecules [FIGS. 1a and 1b) imbedded within glycolipid bilayers in which myelin's flattened oligodendrocytes are also imbedded. Also located on the myelin surface is the oligodendrocyte, topological epitope pairs HSYQE, mimicked by (SEQ ID NO: 2), and VTLRI, mimicked by (SEQ ID NO: 5); and HSYQE plus RNVRF, mimicked by (SEQ ID NO: 4), of myelin oligodendrocyte glycoprotein (MOG) [FIGS. 2 and 3].

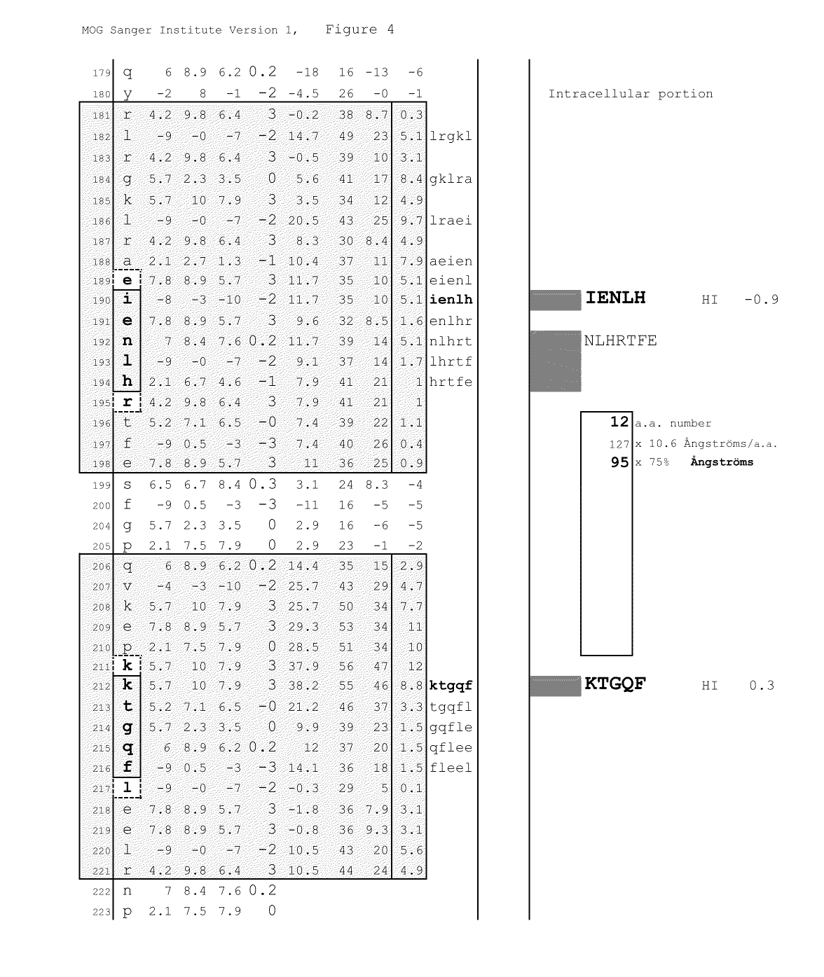

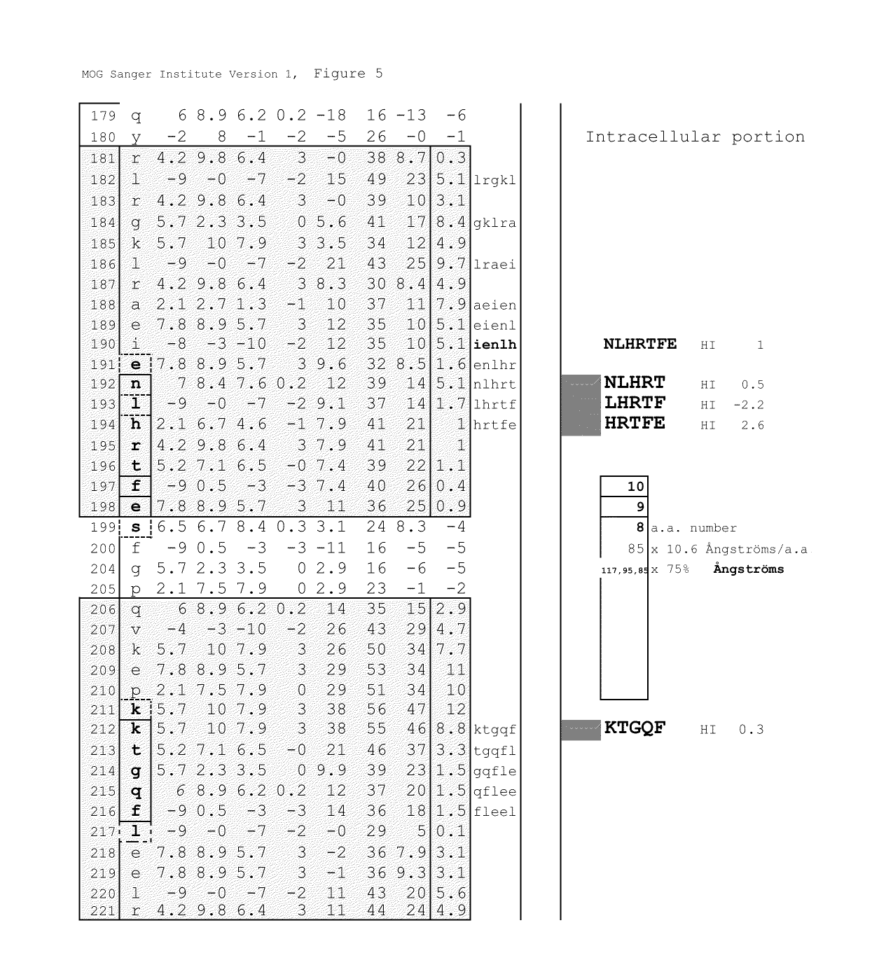

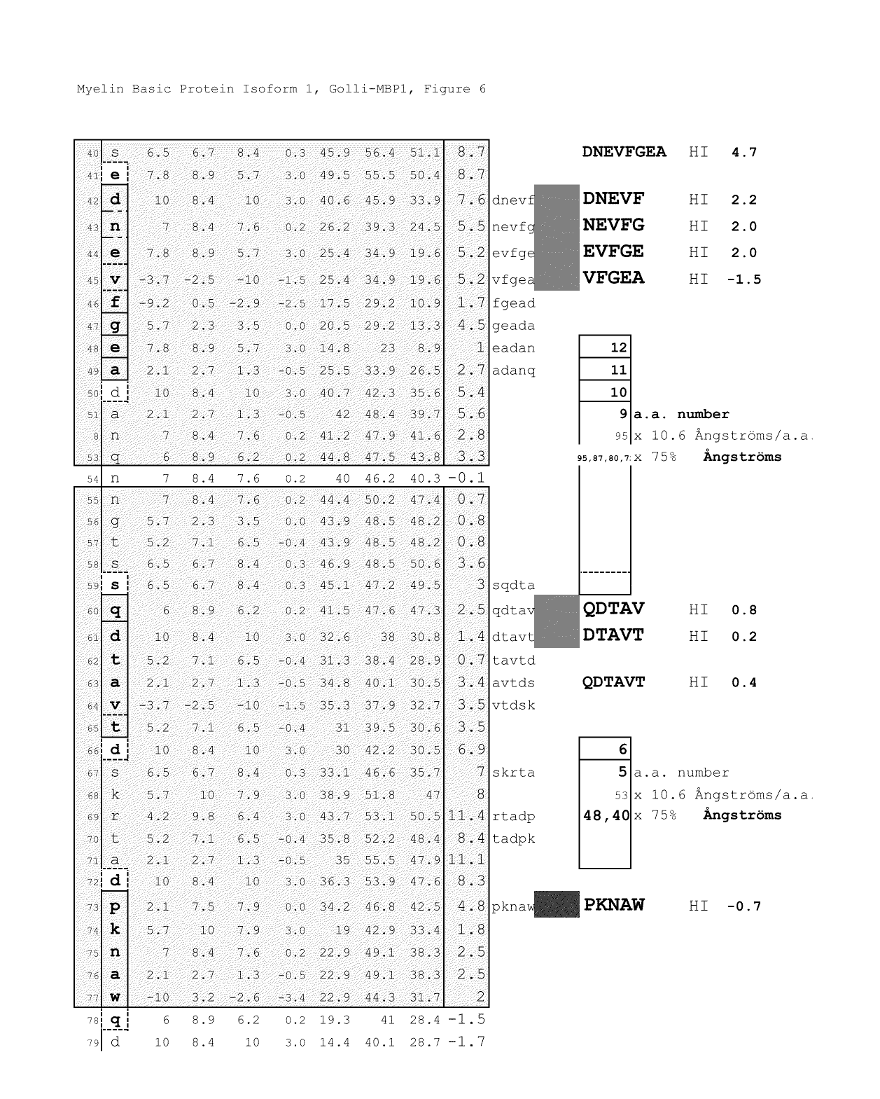

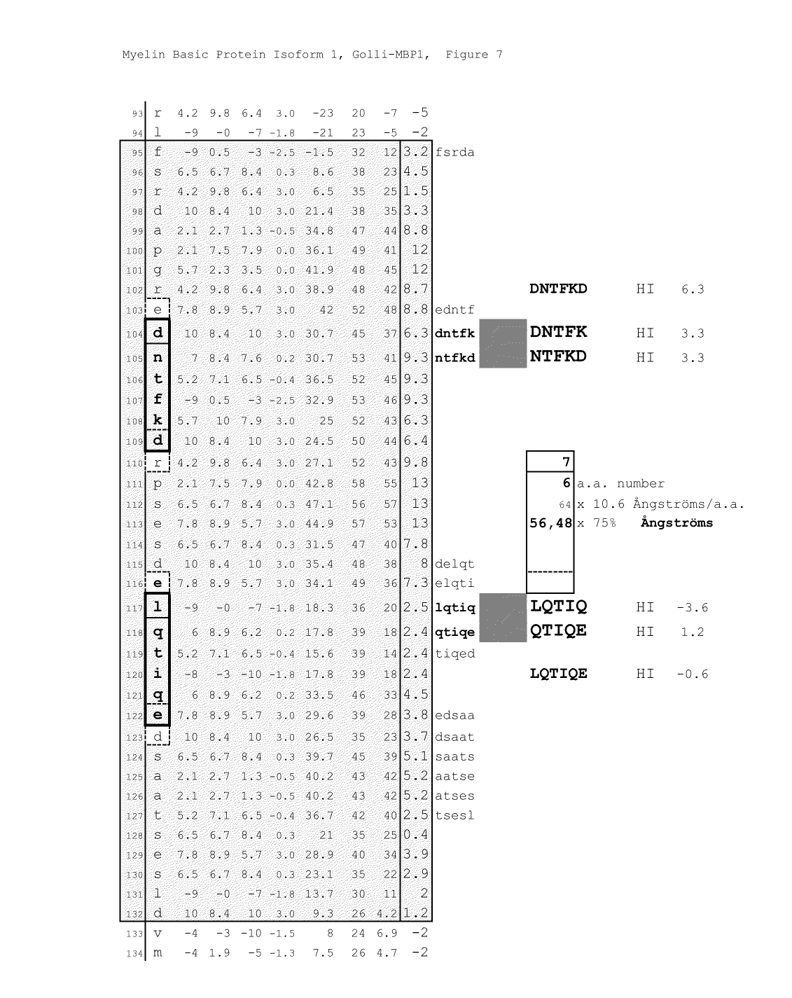

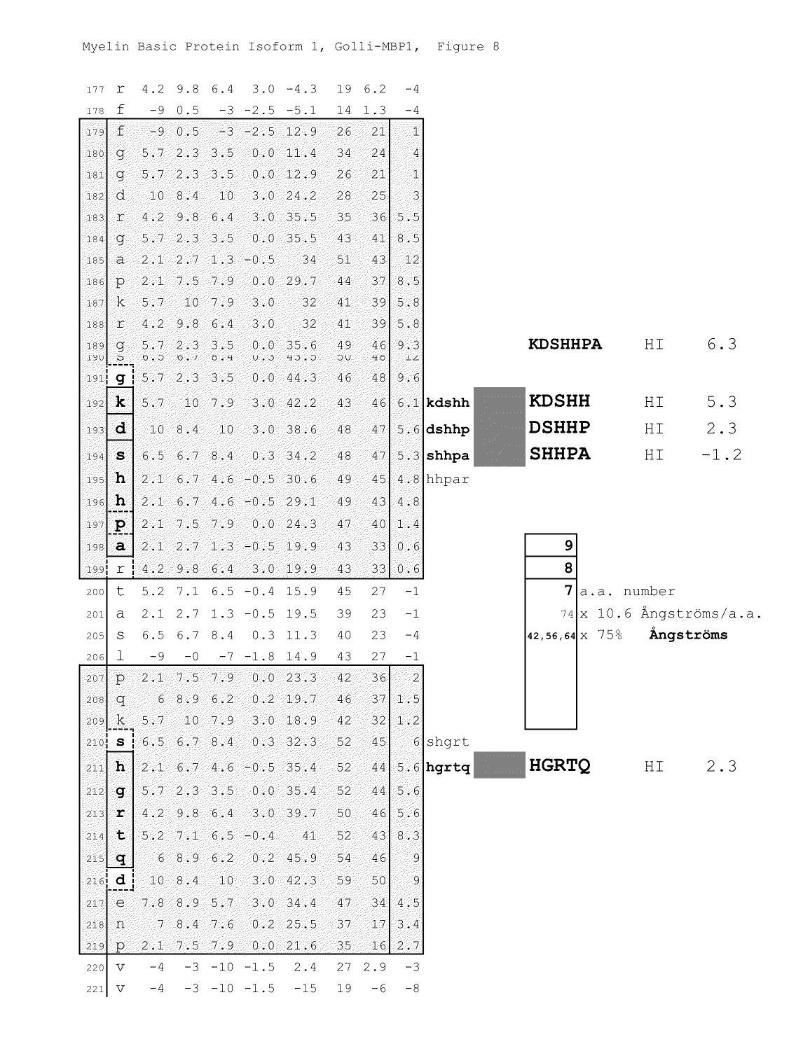

The intra-myelin epitope pairs comprise the (a) intra-myelin MOG epitope pairs KTGQF (SEQ ID NO: 11) and IENLH (SEQ ID NO: 6), KTGQF (SEQ ID NO: 11) and NLHRT (SEQ ID NO: 8), KTGQF (SEQ ID NO: 11) and LHRTF (SEQ ID NO: 9), and KTGQF (SEQ ID NO: 11) and HRTFE (SEQ ID NO: 10) [FIGS. 4 and 5] and myelin basic protein (MBP) epitope pairs QDTAV (SEQ ID NO: 18) and DNEVF (SEQ ID NO: 13), QDTAV (SEQ ID NO: 18) and NEVFG (SEQ ID NO: 14), QDTAV (SEQ ID NO: 18) and EVFGE (SEQ ID NO: 15), QDTAV (SEQ ID NO: 18) and VFGEA (SEQ ID NO: 16), DTAVT (SEQ ID NO: 19) and DNEVF (SEQ ID NO: 13), DTAVT (SEQ ID NO: 19) and NEVFG (SEQ ID NO: 14), DTAVT (SEQ ID NO: 19) and EVFGE (SEQ ID NO: 15), DTAVT (SEQ ID NO: 19) and VFGEA (SEQ ID NO: 16), PKNAW (SEQ ID NO: 20) and QDTAV (SEQ ID NO: 18), PKNAW (SEQ ID NO: 20) and DTAVT (SEQ ID NO: 19), DNTFK (SEQ ID NO: 22) and LQTIQ (SEQ ID NO: 25), NTFKD (SEQ ID NO: 23) and LQTIQ (SEQ ID NO: 25), DNTFK (SEQ ID NO: 22) and QTIQE (SEQ ID NO: 26), NTFKD (SEQ ID NO: 21) and QTIQE (SEQ ID NO: 26), HGRTQ (SEQ ID NO: 28) and YKDSH (SEQ ID NO: 30), HGRTQ (SEQ ID NO: 28) and KDSHH (SEQ ID NO: 31), HGRTQ (SEQ ID NO: 28) and DSHHP (SEQ ID NO: 32), and HGRTQ (SEQ ID NO: 28) and SHHPA (SEQ ID NO: 33) [FIGS. 6 through 11].

A peptide construct for diagnostic purposes can comprise a peptide in total or part from the list ADARM (SEQ ID NO: 1), HSYQE (SEQ ID NO: 2), RNVRF (SEQ ID NO: 4), VTLRI (SEQ ID NO: 5), IENLH (SEQ ID NO: 6), NLHRTFE (SEQ ID NO: 7), KTGQF (SEQ ID NO: 11), DNEVFGEA (SEQ ID NO: 12), QDTAVT (SEQ ID NO: 17), PKNAW (SEQ ID NO: 20), DNTFKD (SEQ ID NO: 21), LQTIQE (SEQ ID NO: 24), HGRTQ (SEQ ID NO: 28), and YKDSHHPA (SEQ ID NO: 29) [Table 4a] wherein the first or last amino acid of each peptide is attached to a hydrophilic linker that is used to couple the peptide construct onto a test surface. The linker may be amino-polyethylene glycol (PEG), amino-8-Fmoc-amino-3,6-dioxa-octanoic acid (amino-ADOOA), amino-8-Fmoc-amino-3,6-dioxa-octanoic acid₂ (amino-ADOOA-ADOOA), or other suitable molecule.

A peptide construct for therapeutic purposes can comprise an individual peptide that is modified, if necessary, for adequate solubility, in-vivo uptake, and/or intravascular distribution without altering its unique epitopic presentation.

Therapeutic short peptides can comprise structurally unique pentamers that have an additional hydrophilic amino acid on either end for enhanced solubility. The additional amino acids represent those that are sequentially present in the constituting myelin protein but confer a peptide sequence structure that that may or not be common to more than one human protein. If found on more than one protein, the commonality in structure may not be an adverse, interfering factor if it is found on protein sections that are either are intracellular and therefore “hidden” from immune system surveillance or, if extracellular, occur on protein regions such as disulfide linkage sites which are also “hidden” and, therefore, immune surveillance-resistant.

The peptide DHSYQE (SEQ ID NO: 3) is an example of a soluble short peptide that is comprised of a singularly unique MOG surface pentamer, HSYQE (SEQ ID NO: 2), but also has an additional amino acid, aspartic acid (D), for enhanced solubility while still remaining singular insofar as the transformed DHSYQ portion is also structurally unique as it does not found on the surface of any other human protein transcribed from the human genome.

The invention provides immunoassays using the mimotopic peptides of the invention. These immunoassays may be used to screen for diseases such as multiple sclerosis. The screening tests are based upon quantification of epitope-specific serum IgE autoantibody as a relative percentage of the total epitope-specific serum autoantibody level.

One such immunoassay is provided to determine the quantity of IgE antibody specific to a single humoral epitope on a myelin protein in a biological fluid sample from a subject comprising (a) contacting the sample with at least one peptide of the invention, (b) determining the amount of IgE antibody bound to the peptide, thereby determining the amount of IgE antibody specific for the myelin protein humoral epitope in the sample. The sample may be biological fluid such as serum.

The subject described in this specification is preferably a human.

The invention also provides a method of diagnosing an autoimmune disorder in a subject comprising performing the described immunoassay, wherein elevated levels of detectable dimeric IgE indicates a diagnosis of an immune disorder, such as MS. Elevated levels of dimeric IgE are defined herein as levels above those of sera from normal, disease-free subjects and depict quantity of individual antibodies that bind to two or more targeted antigenic sites wherein individual sites are interspaced 40-100 Angstroms thus eliciting mast cell degranulation.

Another immunoassay is provided, which is used to measure the relationship between harmful IgE antibodies and competing, protective non-IgE antibodies, comprising quantifying the amount of IgE isotype relative to protective antibody isotypes, and calculating the ratio between these isotypes for an individual myelin epitope.

A method is also provided for treating an immune disorder in a subject comprising administering a composition of the invention to the subject in a therapeutically effective amount and manner sufficient to neutralize a harmful antibody and thereby alleviate the disorder. In a preferred embodiment, the disorder is MS, and the therapeutic of the method suffices to alleviate at least one symptom of MS. The compositions comprising the peptides of the invention neutralizes enough of the damaging epitope-specific IgE antibodies so as to hinder dimer formation from taking place. The composition may be delivered orally, by subcutaneous or intravascular injection, or by topical application. Therapeutic efficacy can be ascertained and/or monitored via interval quantification of biological fluid, epitope-specific IgE and non-IgE antibodies and therapeutic peptide dose adjustment made accordingly.

### EXAMPLES

The following examples are for further explanation of the invention and are not intended to limit the inventions to the specific embodiments.

### Quantification of Ratio of Myelin Epitope Specific IgE to Non-IgE Isotypes.

Serum IgE [4, 59] and Mast cells [5-11] and have been shown to be likely causative and sustaining factors in multiple sclerosis. When coupled to myelin in dimeric form and separated by distances ranging from 40 to 100 Angstroms, projecting IgE is likely to degranulate mast cells [12]. Affected mast cells expel proteolytic enzymes and potentially other factors which damage or destroy targeted myelin and the axons that are sheathed by it.

Epitope-specific IgE is but one isotype involved in the myelin inflammatory process as investigators have also documented the presence of specific IgA, IgG, and IgM [13]. Concomitantly present, the differing isotypes are cross-competitive for epitopic antigen. As a key element of the described invention, an analytical method was therefore developed which quantifies this potential competition and a determination made as to whether the measured competition adequately describes the humoral, MS-specific, autoimmune process.

The analytical method entails quantification of the ratio of myelin epitope-specific IgE divided by the sum of the matching myelin specific non-IgE isotypes. In order to simplify the process, the non-IgE antibody level is determined by measuring epitope-specific human kappa plus lambda chains and subtracting the matching epitope-specific IgE. With experience, it becomes obvious that the specific IgE subtraction is usually unnecessary, as the IgE quantity is exceedingly small in comparison to the matching non-IgE antibodies. Therefore, an evolved MS test can employ the formula: (IgE/(kappa+lambda).

Specific peptides that are 5 amino acids in length mimic individual epitopic structures. These mimotopic peptides represent unique amino acid sequences that are located on the surface of a single, specific myelin protein but on no other human protein transcribed from the human genome.

Materials and Methods Used to Construct and Validate Multiple Sclerosis Test:

Mapping of Dimeric Sites and Derivation of Peptide Constructs:

The Hopp and Woods hydrophilicity method for locating antigenic determinants (epitopic sites) on linear protein sequences [14] is used to predict the humoral epitopes on myelin proteolipid protein (PLP), [15, FIGS. 1a, 1b], myelin oligodendrocyte glycoprotein (MOG), [16, FIGS. 2-5], myelin basic protein [17-19, FIGS. 6-11]

In order to estimate the functional distance (in Ångströms) between epitopes on the surface of each myelin protein as depicted on its Hopp and Woods plot, the following tasks are performed:

The average diameter of constituent amino acids is determined by: (a) comparing the mass of each amino acid relative to the mass of alanine with its known diameter of 6.9 Angstroms [22], (b) multiplying individual mass ratios times 6.9 Ångströms to derive individual estimated amino acid diameters for the non-alanine amino acids, and (c) average the twenty amino acid diameters to obtain an overall average amino acid diameter of 10.6 Ångströms [Table 1].

Individual Hopp and Woods plots are modified so as to depict a protein's hydrophilic surface, either extracellular or intracellular, as if flattened, by trimming away all amino acid regions that are functionally hydrophobic but leaving 2 on each hydrophilic edge to account for in-folding toward the protein center [FIGS. 2 through 11].

The bridging distance between dimeric surface epitopes on individual myelin proteins is estimated by multiplying the intervening amino acid number by 10.6 Ångströms per amino acid.

Because a protein surface is not flat but oscillates in depth, sera from an age and gender varied negative control group are tested sequentially while reducing the estimated dimeric distances in 5 percent intervals to find a reduction percentage which simulated staircase dips in normally rolling surface contours of proteins and also afforded functionally negative test results for the controls. A 25% reduction of the linear distances attained in step 3 between epitopes affords test-negative results for all trial-tested control serum samples [59].

Pathological dimer bridging values and locations are depicted in the FIG. 2 through 11 for myelin oligodendrocyte glycoprotein and myelin basic protein.

A similar contour-mapping approach is not used for myelin proteolipid protein (PLP) because of its studded presence in myelin lamellae. PLP is numerously expressed and imbedded in glycolipid lamellae wherein one PLP molecule is displayed in one lamella and another PLP expressed in a second, 65-71 Ångströms distal to the first [23]. Proteolipid monomers and their inclusive ADARM epitopes, therefore, serve as ideal IgE dimer-binding sites. A PLP-positive IgE/(kappa+lambda) test result is, therefore, an indication of a respective, functional dimer-positive presence.

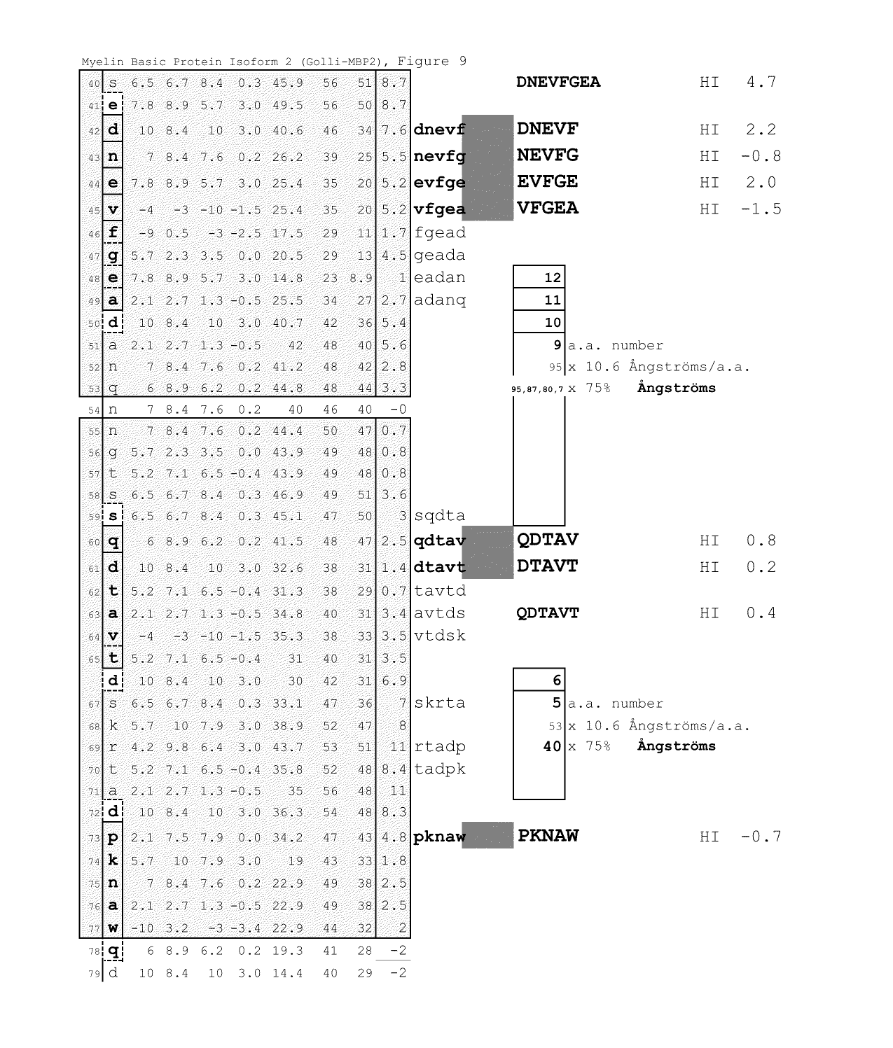

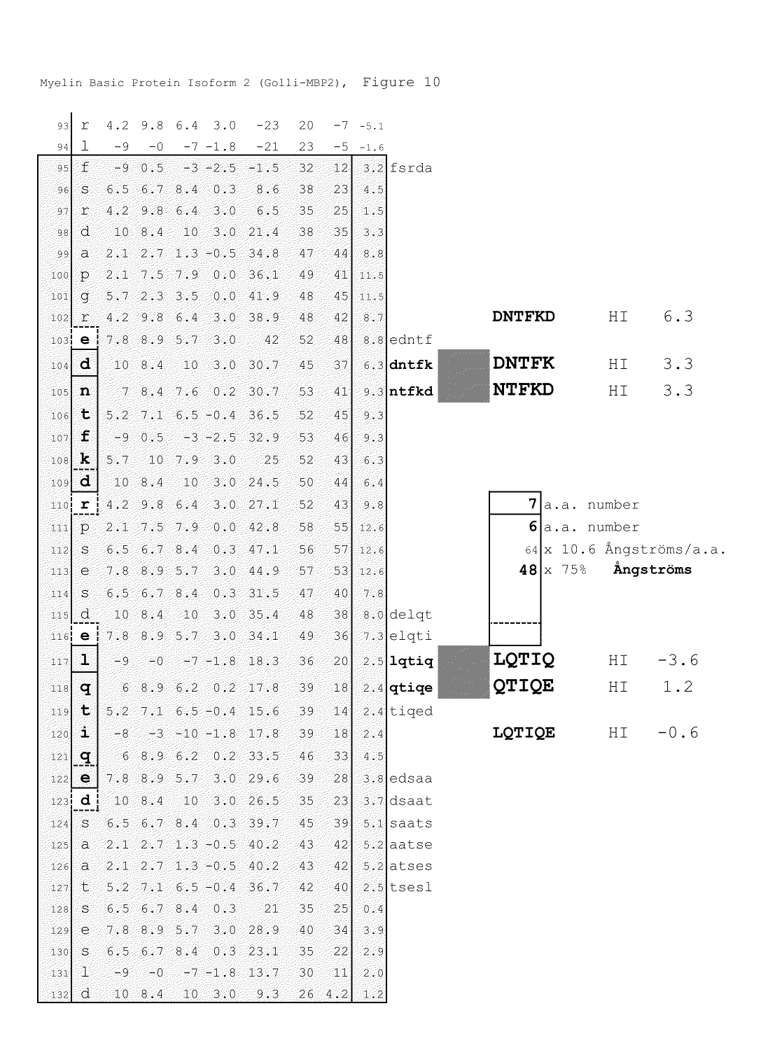

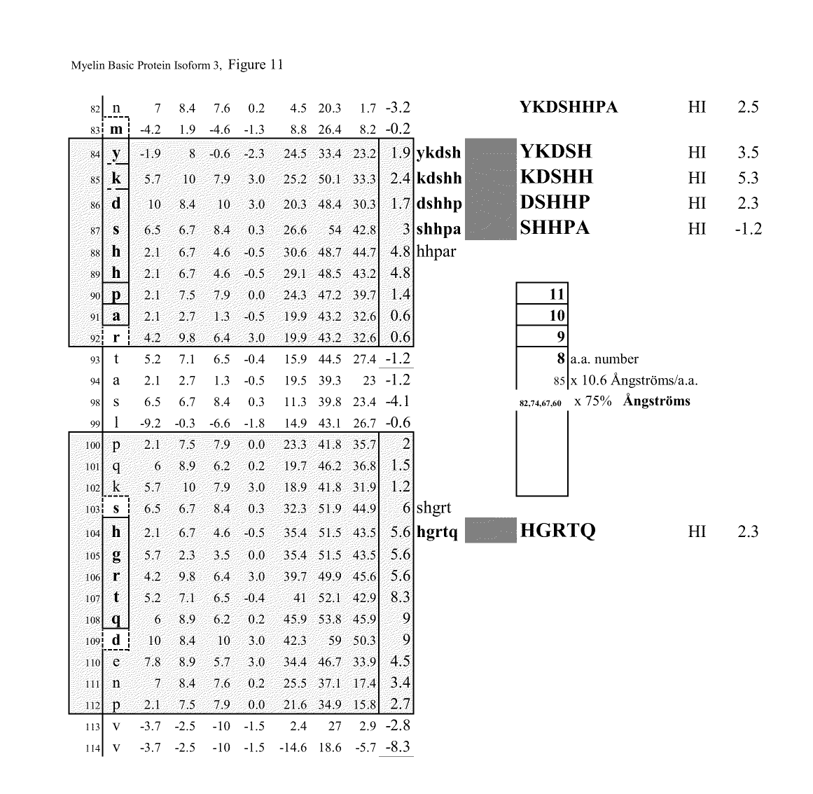

MOG has been shown to be differentially expressed in various isoforms. However, for the purpose of identifying the potential array of MOG humoral epitopes possible on all isoforms and the epitopes' utility in dimer formation, analysis of MOG Sanger Institute Isomer 1 [FIGS. 2 through 5] proves sufficient. The analysis illustrates the presence of two likely, disease-functional dimers expressed on the portion of MOG that is expressed on the myelin (oligodendrocyte) surface and four potential subsurface (intracellular) dimers if the latter were somehow exposed by myelin surface disruption. Similar subsurface access would expose potential myelin basic protein (MBP) dimer sites exemplified by Hopp and Woods plots of myelin basic protein isoform 1 [FIGS. 6, 7, 8], Isoform 2 [FIGS. 9, 10], and Isoform 3 [FIG. 11].

Test Components.

Microtiter Test Plate Layout.

96-well maleic anhydride-activated microtiter plates (i.e. Thermo Scientific) are used. Each peptide construct solution corresponding to the listed pentamers is applied at 100 μL/well into 4 consecutive vertical wells. Four consecutive vertical wells per plate are left blank for determining background.

Each peptide construct applied to the microplate wells consists of a mimotopic peptide preceded by an aminated hydrophilic linker, 8-amino-3,6-dioxaoctanoic acid8-amino-3,6-dioxaoctanoic acid₂ as custom-synthesized by Mimotopes Pty, Clayton, Australia or other peptide construct providers. Peptide tertiary lysine amino groups are protected with a (4,4-dimethyl-2,6-dioxocyclohex-1-ylidene)-3-methylbutyl (ivDde) blocking group to prevent inadvertent lysine tertiary amine binding when attaching peptide constructs to test plate wells.

Peptide Constructs Formulation:

Each construct is dissolved in pH 7.2 phosphate buffered saline (PBS) immobilization buffer (Product No. 28372, Thermo Scientific, Rockford, Ill., USA) in an assay-optimum μg/mL concentration [Table 2].

Peptide Constructs Application:

100 μL of each peptide construct solution is applied in quadruplicate to 96-well amine-binding, maleic anhydride-activated white 96-well plates (Product No. 15108, Thermo Scientific) or similar test plates. Four wells are left blank per plate for background determination. The plates are covered with acetate plate sealers (Thermo Scientific, Boston, Mass. Product No. 3501) and the construct solutions incubated at 21-26 degrees C. for 18-24 hrs.

Plate Blocking Procedure:

The peptide construct solutions are aspirated and 120 μL of HSA blocking solution (10 mg recombinant human serum albumin per mL immobilization buffer) applied per well. The plates are covered with acetate plate sealers and incubated at 21-26 degrees C. for 18-24 hrs. and then aspirated and dried.

Lysine Unblocking Procedure:

200 μL of 2% hydrazine monohydrate (Sigma Chemical Company, St. Louis, Mo., Product 207942) in DMSO (dimethyl sulfoxide, Thermo Scientific product #20688) is applied per well and incubated for 10 minutes. The hydrazine solution is aspirated and the procedure repeated two additional times. 250 μL of phosphate buffered saline with 0.05% Tween-20 (PBST, Thermo Scientific Product #28320) is applied per well. Plates are incubated for 30 minutes and aspirated.

Microplate Storage:

After drying, test plates are sealed with acetate plate sealers and stored at room temperature until needed. Individual plates were used for both specific IgE and specific (kappa+lambda) assays.

MS Test Procedures: Specific IgE Portion.

The specific IgE immunoassay entails use of 100 μL/well of neat subject serum that has been spiked with 1 mg/mL amino-ADOOA-ADOOA linker (50 μL linker solution per 12 mL serum). Plates are sealed and incubated for 2 hours at 21-26 degrees C. and then washed with PBST. 100 uL of 4 μg/mL biotinylated goat anti-human IgE (96 μL of Vector Labs, Burlingame, Calif., USA product No. BA-3040 per 11.9 mL of conjugate diluent (10 mg/mL recombinant HSA in PBST+0.25% PEG 4000)) is applied per well. After 2 hours incubation, the plates are washed and 100 uL/well of 64 ng/mL streptavidin horseradish peroxidase (ThermoPierce Product No. 21126 diluted in HSA conjugate diluent) is applied. Test plates are incubated for 30 minutes and then washed. 100 μL/well of ThermoPierce chemiluminescence substrate (product No. 37074) is applied and the plate(s) read 1-3 minutes post application using a microplate luminometer such as the Luminoskan Ascent Microplate Luminometer (Thermo Fisher Scientific, Waltham, Mass., USA). Test plates are incubated for 30 minutes and then washed. 100 μL/well of ThermoPierce chemiluminescence substrate (product No. 37074) is applied and the plate(s) read 1-3 minutes post application using a microplate luminometer such as the Luminoskan Ascent Microplate Luminometer (Thermo Fisher Scientific, Waltham, Mass., USA).

MS Test Procedures: Specific Kappa+Lambda Portion.

The linker-spiked test serum sample used in the specific IgE assay is diluted 1/25,000 by: (a) making a 1/100 dilution via co-mixture of 100 μL serum and 9.9 mL PBST and (b) spiking 11.950 mL of HSA conjugate diluent with 48 μL of the 1/100 diluted serum. Plates are filled with 100 μL/well of diluted serum, sealed, and incubated for 2 hours at 21-26 degrees C. Equal volumes of Vector biotinylated, goat anti-human kappa antibody (BA-3060) plus biotinylated, goat anti-human lambda antibody (BA-3070) are mixed together to form a biotinylated anti-K+L concentrate (500 μg/mL). 96 μL of the anti-K+L concentrate is mixed with 11.9 mL of HSA conjugate diluent and 100 μL of the resulting solution applied per well. After 2 hours incubation, plates are washed and 100 μL per well of 16 ng/mL streptavidin horseradish peroxidase solution applied. Test plates are incubated for 30 minutes, aspirated, and washed. 100 uL/well of ThermoPierce chemiluminescence substrate is applied and the plates read at 1-3 minutes post application.

IgE/(Kappa+Lambda) Determination.

Specific IgE and matching specific K+L signals are obtained by reading corresponding test plates on the microplate luminometer. An average test value corresponding to individual mimotopic peptides is determined by discarding the highest and lowest of four values and averaging the remaining two. The same is done for the four background well values plus calculation of twice the standard deviation of the two-point average. The blank well background is deemed to be its average value plus twice the standard deviation. Each peptide-coated well average is subtracted by the plate background value to yield a net signal. K+L values are multiplied by 25,000 in order to delineate the corresponding neat serum (undiluted) epitope-specific K+L antibody level. IgE/(K+L) values are multiplied by 1,000,000 in order to bring each to a positive whole number wherever possible. Test results with net negative values or values less than 0.5 are assumed to be test-negative.

Examples of Expected MS Test Results.

MS-Negative Control Subject Results.

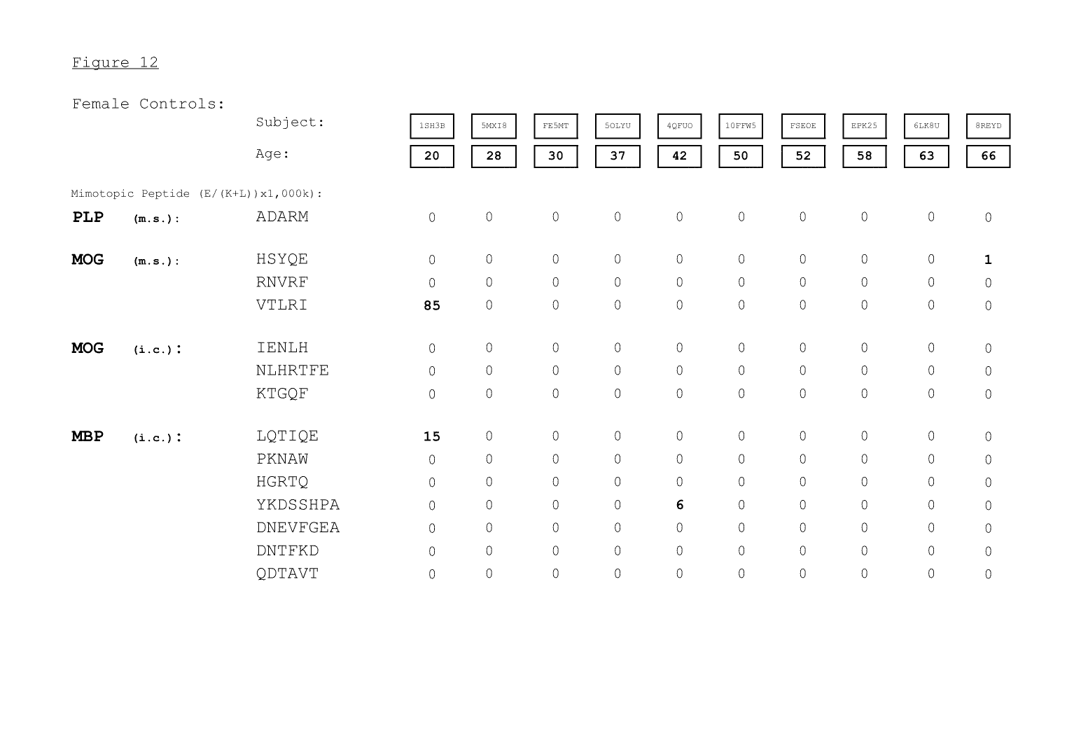

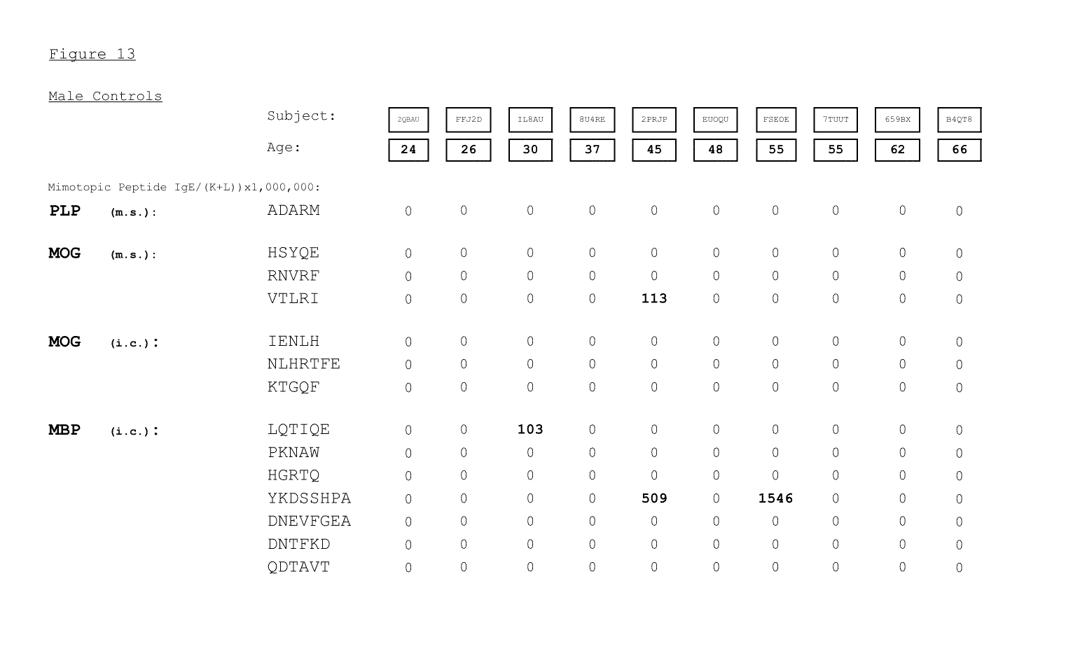

As depicted in FIGS. 12 and 13, MS-positive test results are, at best, confined toward attainment of IgE/(kappa+lambda)-positive values against single, non-dimer participating epitopes.

MS Patients not Receiving Interferons, Copaxone, or Immunoassay-Altering Medications.

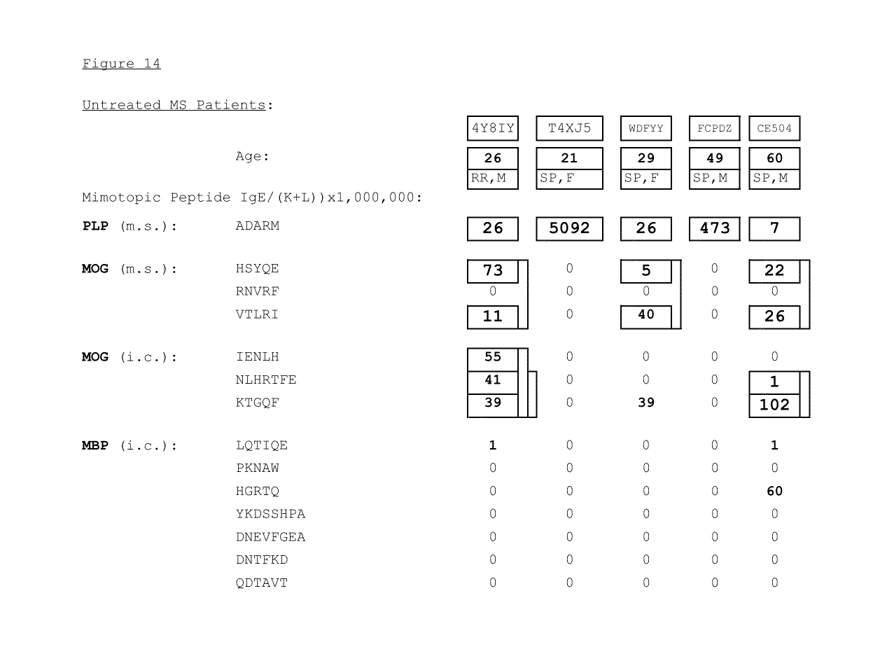

As depicted in FIG. 14, previously untreated patients should all be dimer test-positive against the PLP epitope ADARM and also, possibly, against the MOG surface and subsurface epitopic dimers. Test results should be distinct and fairly robust.

MS Patients Treated with Interferon(s) and/or Copaxone.

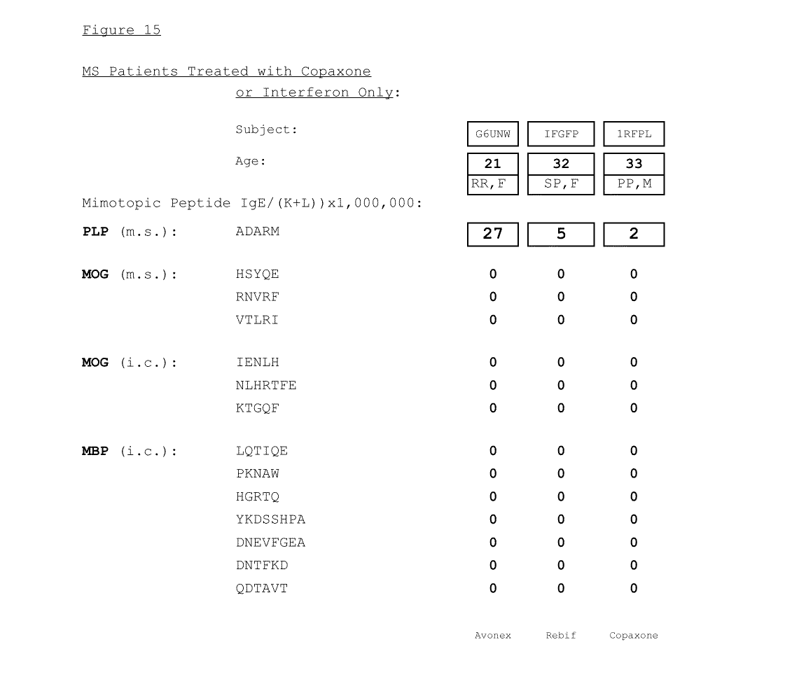

As depicted in FIG. 15, such patients may be weakly test positive against dimeric myelin epitopic peptide ADARM because of varying degrees of expected humoral immune suppression.

MS Patients Treated with Interferon(s) and Potentially Immunoassay-Altering Medication(s).

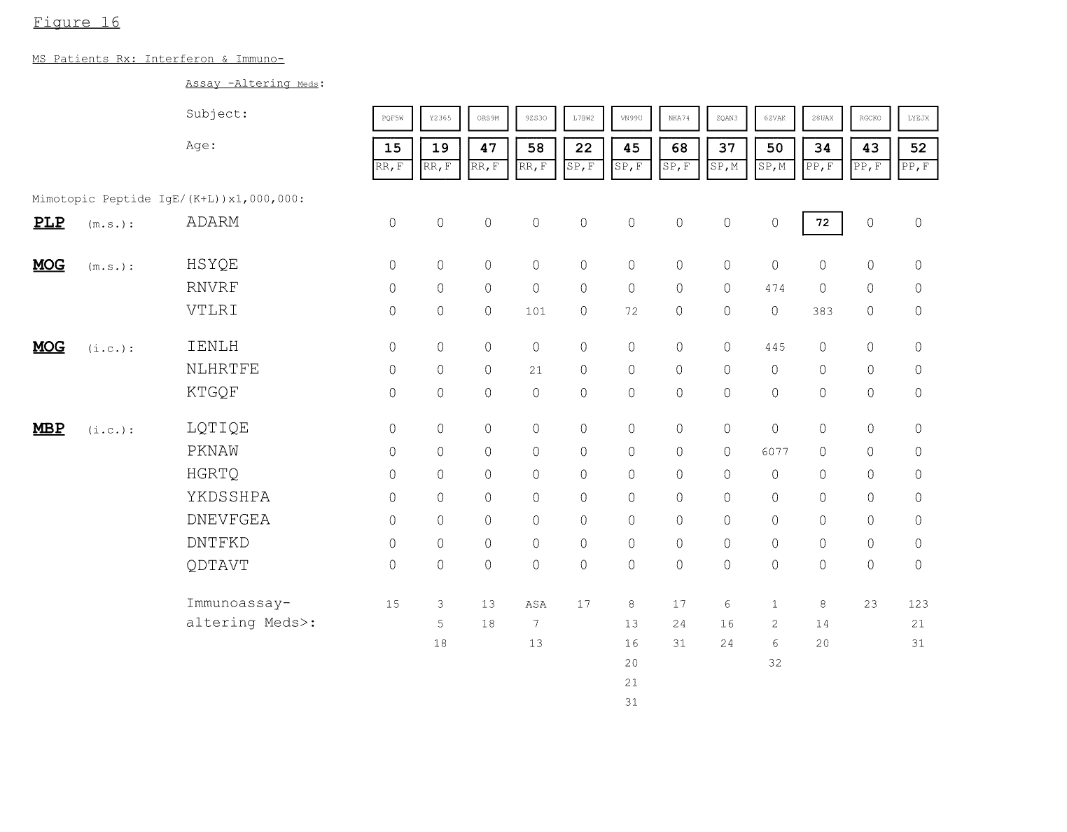

As depicted in FIG. 16, such patients may only sporadically be test positive because of immunoassay inference by psychotropic pharmaceuticals and other medications (59).

MS Patients Treated with Potentially Immunoassay-altering Medication(s) Only.

Such patients may be sporadically test-positive against the dimeric myelin epitopic peptide ADARM because of immunoassay inference by psychotropic pharmaceuticals and other medications [59].

Patients Treated with Copaxone and Potentially Immunoassay-Altering Medication(s).

Such patients may be sporadically test-positive against dimeric myelin epitopic peptide ADARM and also the MOG surface dimeric epitopes because of immunoassay inference by psychotropic pharmaceuticals and other medications [59].

Example of a Formulated Peptide-Based MS Therapeutic and Pilot Clinical Trial.

It has been observed that multiple sclerosis (MS) is caused or adversely affected by unencumbered, myelin-specific IgE autoantibodies that trigger focal mast cell degranulation with release of tissue-damaging enzymes and other factors. A peptide-based therapeutic was, therefore, formulated to neutralize the damaging antibodies and halt or diminish the degranulation, and thereby reverse the MS process.

A mixture of three mimotopic peptides simulating targeted myelin epitopes was formulated for subcutaneous injection: (1) ADARM (SEQ ID NO: 1) is the amino acid structural homolog of the lone, but multivalent proteolipid protein humoral epitope and (2) DHSYQE (SEQ ID NO: 3) and KTGQF (SEQ ID NO: 11), each structurally representing a dimer cornerstone epitopes of myelin oligodendrocyte glycoprotein (MOG). Cornerstone herein is defined herein as being an epitope that is common to two or more dimers on the surface of a molecule. Elimination of a specific IgE binding to a cornerstone epitope would therefore prevent mast cell degranulation from taking place for two or more dimeric conditions [FIGS. 2, 3, 4, and 5].

A single patient, pilot clinical trial was undertaken to gauge the likelihood of therapeutic efficacy. In order to completely eliminate in-vivo, epitope-specific IgE and other isotypes, an estimate was made of the mixture quantity needed weekly to provide complete daily autoantibody neutralization.

Optimum Dose Implementation of the Three-Peptide MS Therapeutic Comprised:

(a) estimating the molar quantity of in-vivo, epitope specific IgE plus non-IgE antibody requiring in-vivo neutralization; (b) discerning the negative effect of renal and hepatic clearance upon administered peptides; (c) composing a 50% glycerol mixture of the peptides ADARM (SEQ ID NO: 1), DHSYQE (SEQ ID NO: 3), and KTGQF (SEQ ID NO: 11); and (d) commencing a reasonable injection schedule monitored by interval, measurements of IgE/(kappa+lambda) levels against the myelin surface target epitopes in order to discern a significant reduction or eliminations of their harmful, epitope-specific IgE autoantibodies.

Estimating Epitope-Specific Antibodies Requiring Neutralization.

For patients who were serum IgE/(kappa+lambda) test-positive against PLP and MOG dimers, the estimation process entailed the following steps:

Determining the maximum quantity per milliliter quantity of IgA, IgE, IgG, and IgM in the average human male [Table 5];

Converting respective serum antibody levels to gram per milliliter [Table 5];

Converting gram per milliliter to moles per mL serum using the formula, mole=gram weight of sample/relative molar mass [Table 6];

Multiplying individual antibody mole times antibody valence (isotype antigen binding sites) [Table 6];

Multiplying each isotype-specific mole valence/mL value times 2,750 mL which is the average U.S. adult male total serum volume), [Table 6];

Dividing each isotype-specific value by 1,000,000 possible, discernible, humoral epitopes (subjective starting point) and then summating in order to estimate moles of mimotopic peptide needed to completely neutralize all individual, single epitope-specific, in-vivo antibodies (summated molar value=0.0000000060164) [Table 7];

Determining the collective molar mass of the 3 anterior, MOG cornerstone-epitope amino acid sequences assuming that KTGQF will omprise a two-fold representation because of its dual molecule aggregate-forming tendencies [Table 8];

Ascertaining the weight of the 3 anterior myelin, epitope-neutralizing peptides by multiply the combined peptides' molar mass (2424.64) times the single epitope-specific antibodies' summated molar value (0.0000000060164) in order to yield a net value of 0.000014587604 grams; and

Multiply 0.000014587604 grams by 1,000,000 μg/gram to attain 14.586 micrograms of total epitope specific antibody neutralizing peptides mixture required per day. (If needed, one could repeat steps g through i for the three intracellular cornerstone mimotopic peptides exemplified in Table 9 for myelin basic protein).

Mimotopic Peptide Formulation.

The Three-peptide MS therapeutic was formulated by:

[Table 8] mixing together 213.8 mL of 50% pharmaceutical grade glycerin (Allergy Laboratories, Inc., Oklahoma City, Okla. U.S.A.) plus the 99 percent pure peptides (Polypeptide Laboratories, San Diego, Calif., USA): ADARM (SEQ ID NO: 1), (487 mg); DHSYQE (SEQ ID NO: 3), (651 mg); and KTGQF (SEQ ID NO: 11), (1,000 mg). The peptides were shaken into solution and the solution and sterile-filtered through Pall PN 4902 Supor EKV 0.2 μm filters (Pall Corporation, Ann Arbor. Mich., U.S.A.).

Injection Schedule.

The weekly time interval between injections was established by: (a) formulating an estimated peptide renal clearance/enzymatic degradation timetable that would be expected to reflect subcutaneous injection of 40 mg of tri-peptide/50% glycerin solution followed by an anticipated immediate commencement of a 10 minute intravascular peptide half-life [Reference 11, Table 10]; (b) ascertaining that one week as a reasonable, initial time interval for injection administration; and (c) performing peptide-specific serum IgE/(kappa+lambda)×1,000,000 (1) determinations at 1, 2, 4, and 8 weeks post-therapy commencement to monitor specific IgE reduction/elimination efficiency.

Assessing Therapeutic Efficacy by Measuring Epitope-Specific Serum Autoantibody Levels.

Analysis of serum samples obtained at 1, 2, 4 and 8 weeks post therapy initiation revealed no evidence of remaining epitope-specific IgE or non-IgE serum antibodies against the ADARM (SEQ ID NO: 1) and HSYQE (SEQ ID NO: 2) myelin surface epitopes [Reference 67 and FIG. 12b] implying diminution or cessation of myelin-targeted mast cell degranulation and resulting pathology.

Assessing Therapeutic Efficacy via Post-Treatment Medical Examination.

The pilot study patient's clinical status had improved following initiation of tri-peptide therapy. Specific changes included: (a) improved balance when walking and (b) recovered sensation in his right foreleg (previously absent for 18 years following an early stage MS relapse); and (c) development of a non-irritating nor pathological hyperosmia to standard kitchen odors as well as mild, non-troubling hyperacusis.

### CONCLUSION

Product development and validation test data indicates that multiple sclerosis is a humoral autoimmune disease caused by IgE dimer formation on the surface or immediate subsurface of CNS myelin that results in focal mast cell degranulation. The degranulating mast cells release proteolytic enzymes and possibly other factors that damage or destroy proximal neurons.

Test results of MS patients taking medication shown to have an immunosuppressive effect suggest either interference with the normally expected IgE/competing antibody, pathological process or with the MS test itself. This is inferred by the quantitative difference in MS test scores between the patients who take no medication and exhibit relatively high test scores, patients who are being successfully treated with a single MS-specific pharmaceutical, beta interferon or Copaxone, but have relatively low positive test scores, and patients who are receiving agents shown to be immunosuppressive and are only sporadically MS test-positive,

One's understanding of the factors involved in the MS process is hindered by the singular or multiplex incorporation of therapeutic substances other than beta interferon or Copaxone that are concomitantly given to patients or prescribed alone.

As interferon and Copaxone are prescribed because of their MS-immunosuppressive therapeutic effect and MS is a likely autoimmune disease, it is reasonable to expect some decrease in humoral immune function among specifically treated patients as reflected by the lower test scores exhibited in FIG. 15. However, the difference exhibited by MS patients who are also receiving ancillary therapeutic substances that are additionally immunosuppressive suggests that an augmentation of immune suppression may not always be therapeutically beneficial nor escape MS serum test interference.

Also suggested is the possibility that the ancillary medications, having only been in use since the early twentieth century, may have historically played an instigating or promoting role in MS causation and/or progression, through disruption of homeostatic immune system controls.

The absence of MS test-positive IgE/(kappa+lambda) results against MBP dimers may be due to dimers' deep, intracellular location and freedom from immune surveillance and pathological action or may indicate that myelin basic protein is not the principal autoimmune target in multiple sclerosis. It may also suggest that it is an autoimmune target but of lesser frequency or importance than the epitopes on the outer surface or immediate sub-surface of myelin.

As untreated MS appears to be an IgE dimer-driven, humoral autoimmune disease, as is suggested by the available test data, treatment with mimotopic peptides homologous to those used in the immunoassay are likely to prove therapeutically effective by neutralizing anti-myelin IgE antibodies and slowing down or stopping mast cell degranulation.

The therapeutic peptides need to be administered in such a way as to insure intravascular delivery of quantities sufficient to neutralize most epitope-specific dimeric IgE autoantibodies via antibody-to-peptide complexing or by neutralizing one key epitope-specific IgE antibody whose target epitope is a cornerstone of a number of pathological dimers, the absence of which would abrogate the pathological process. Such singular, dimer-blocking peptides are listed in the right-hand column of Table 4a and whose corresponding epitopes are exhibited in FIG. 2 through 11.

Therapeutic peptides need possess: (a) an exact structural match with the specific myelin protein epitope; (b) be of sufficient length (5-7 amino acids) to comfortably fit and be avidly bound by a single autoantibody; (c) be relatively hydrophilic so as to be functionally soluble when injected or ingested; and (d) if ingested be aided in their enteric absorption by pharmaceutical agents such as medium-chain fatty acid constructs (56), and/or super porous hydrogels (57), and/or N-trimethyl chitosan chloride (58).

### LIST OF REFERENCES

The following are incorporated by reference to the extent they relate materials and methods of the present disclosure. [1] GöpelW. Immunosuppressive therapy of multiple sclerosis using cyclophosphamide and imuran report on 57 cases. Dtsc Gesundheitsw1972; 27:1955-11961. [2] Jacobs L. Intrathecal interferon reduces exacerbations of multiple sclerosis. Science 1981; 214:1026-1028. [3]Berger J R. Functional improvement and symptom management in multiple sclerosis: clinical efficacy of current therapies. Am J Manage Care 2011; 5:S146-153. [4] Mikol D D, Calenoff E, et al. Serum IgE reactive against small myelin protein-derived peptides is increased in multiple sclerosis patients. J Neuroimmunol 2006; 180: 40-49. [5] Secor V H, Brown M A, et al. Mast cells are essential for early onset and severe disease in a murine model of multiple sclerosis. J. Exp. Med. 2000; 191: 813-822. [6]Brown M A, Tanzola M B, Robbie-Rya n M. Mechanisms underlying mast cell influence on EAE disease course. Molec Immunol 2002; 38: 1373-1378. [7]Medic N, Lorenzon P, Vita F, Trevisan E, et al. Mast cell adhesion induces cytoskeletal modifications and programmed cell death in oligodendrocytes. J Neuroimmunol 2010; 218: 57-66. [8]Ibrahim M Z M. The mast cells of the mammalian central nervous system. The effect of proton irradiation in the monkey. J Neurological Sciences 1974; 21: 479-499. [9]Dietch, G. N., Hinrichs, D. J., 1991. Mast cell proteases liberate stable encephalitogenic fragments from intact myelin. Cellular Immunol. 135, 541-548. [10] Johnson D, Seeldrayers P A, Weiner H L. The role of mast cells in demyelination. 1. Myelin proteins are degraded by mast cell proteases and myelin basic protein and P2 can stimulate mast cell degranulation. Brain Research 1988; 444: 195-198. [11]Ibrahim M Z, Reder A T, et al. The mast cells of the multiple sclerosis brain. J Neuroimmunol 1996; 70: 131-138. [12] Paar J M, Harris N T, et al. Bivalent ligands with rigid double-stranded DNA spacers reveal structural constraints on signaling by Fc epsilon R I. J Immunol 2002; 169:856-864. [13] Newcombe J, Gahan S, Cuzner M L. Serum antibodies against central nervous system proteins in human demyelinating disease. Clin Exp Immunol 1985; 589: 383-390. [14]Hopp T P, Woods K R. Prediction of protein antigenic determinants from amino acid sequences. Proc Natl Acad Sci USA 1981; 78(6): 3824-3828. [15]Kronquist K E, Crandall B F, et al. Expression of myelin proteins in the developing human spinal cord: cloning and sequencing of human proteolipid protein cDN A. J. Neurosci. Res 1987; 18:395-401. [16]Sanger 1. Myelin oligodendrocyte glycoprotein isoform CAQ08210 amino acid sequence submitted on Jan. 9, 2009 by the Wellcome Trust Sanger Institute, Hinxton, U.K. MBP Isoform 1: Eylar E H, Brostoff S., et al. Basic A1 protein of the myelin membrane. The complete amino acid sequence. J Biol. Chem. 1971; 246(18); 5770-5784. [18]MBP Isoform 2: Eylar E H. as per MBP Isoform 1 minus amino acids 193-304. [19]MBP Isoform 3: Eylar E H.; as per MBP Isoform 1 minus amino acids 1-133 and amino acid 192 is substituted by KVPWLKPGRSPLPSHARSQPGLCNMYK. [20] Mikol D D, Gulcher J R, et al. The oligodendrocyte-myelin glycoprotein belongs to a distinct family of proteins and contains the HN K-1 carbohydrate. J Cell Biol 1990; 110: 471-479. [21]Bronstein J M, Tiwari-Woodruff S, et al. Involvement of OSP/claudin-11 in oligodendrocyte membrane interactions: role in biology and disease. J Neurosci Res 2000; 59: 706-711. [22]Hashizume H, Theng B K G, et al. Adsorption and discrimination of alanine and alanyl-alanine enantiomers by allophone. Clay Minerals 2002; 37: 55 1-557. [23]Brown FR 3rd, Kirschner D A, et al. X-ray diffraction analysis of myelin lipid/proteolipid protein multilayers. J Neurosci Res 1989; 24: 192-200. [24]Wahl C, Liptay S, Adler G, et al. Sulfasalazine: a potent and specific inhibitor of nuclear factor kappa B. J Clin Invest 1998; 101: 163-174. [25]Badawy A H, Shalaby S A, et al. Hydantoin immunosuppression clinical study. J Egypt Soc Parasitol 1991; 21: 257-262. [26]Yamada M, Ohkawa M, et al. Anticonvulsant-induced suppression of IFN-gamma production by lymphocytes obtained from cervical lymph nodes in glioma-bearing mice. J Neurooncol 2000; 47: 125-132. [27]Sugino H, Futamura T, et al. Atypical antipsychotics suppress production of proinflammatory cytokines and upregulate interleukin-10 in lipopolysaccharide-treated mice. Neuropsychopharmacol Biol Psychiatry 2008; 33: 303-307. [28]Chang M P, Castle S C, et al. Suppressive effects of alprazolam on the immune response of mice. Int J Immunopharmacol 1991; 13: 259-266. [29]Sorrell T C, Forbes I J. Depression of immune competence by phenyloin and carbamazepine. Studies in vivo and in vitro. Clin Exp Immunol 1975; 20: 273-2 85. [30] D'Ambrosia A, Segoloni G, et al. The modulatory effect of diltiazem on human in vitro alloreactivity when used alone or in combination with cyclosporine A and/or methylprednisolone. Transpl Int 1997; 10:426-431. [31]Wey S P, Wu H Y, et al. Methamphetamine and diazepam suppress antigen-specific cytokine expression and antibody production in ovalbumin-sensitized BALB/c mice. Topical Lett 2008; 181: 157-162. [32]Paul F, Waiczies S, et al. Oral high-dose atorvastatin treatment in relapsing-remitting multiple sclerosis. 2008; PLoS One 3(4): e1928. [33]Delayre-Orthez C, Beck er J, et al. Suppression of allergen-induced airway inflammation and immune response by the peroxisome proliferator-activated receptor-alpha agonist fenofibrate. Eur J Pharmacol 2008; 581: 177-184. [34]Kobashigawa J A, Laks H, et al. The University of California at Los Angeles experience in heart transplantation. Clin. Transpl. 1995; 1995: 129-135. [35]Schmidt W M, Spiel A O, et al. In-vivo effects of simvastatin and rosuvastatin on global gene expression in peripheral blood leucocytes in a human inflammation model. Pharmacogenet Genomics 2008; 18: 109-120. [36]Kim Y C, Kim K K, et al. Simvastatin induces Fox p3+T-regulatory cells by modulation of transforming growth factor-beta signal transduction. Immunology 2010; 130: 484-193. [37] Devo ino L, Idova G, et al. Brain neuromediator systems in the Res 2000; 59: 706-711. [22]Hashizume H, Theng B K G, et al. Adsorption and discrimination of alanine and alanyl-alanine enantiomers by allophone. Clay Minerals 2002; 37: 55 1-557. [23]Brown F R 3rd, Kirschner D A, et al. X-ray diffraction analysis of myelin lipid/proteolipid protein multilayers. J Neurosci Res 1989; 24: 192-200. [24] Wahl C, Liptay S, Adler G, et al. Sulfasalazine: a potent and specific inhibitor of nuclear factor kappa B. J Clin Invest 1998; 101: 163-174. [25]Badawy A H, Shalaby S A, et al. Hydantoin immunosuppression clinical study. J Egypt Soc Parasitol 1991; 21: 257-262. [26] Yamada M, Ohkawa M, et al. Anticonvulsant-induced suppression of IFN-gamma production by lymphocytes obtained from cervical lymph nodes in glioma-bearing mice. J Neurooncol 2000; 47: 125-132. [27]Sugino H, Futamura T, et al. Atypical antipsychotics suppress production of proinflammatory cytokines and upregulate interleukin-10 in lipopolysaccharide-treated mice. Neuropsychopharmacol Biol Psychiatry 2008; 33: 303-307. [28]Chang M P, Castle S C, et al. Suppressive effects of alprazolam on the immune response of mice. Int J Immunopharmacol 1991; 13: 259-266. [29]Sorrell T C, Forbes I J. Depression of immune competence by phenyloin and carbamazepine. Studies in vivo and in vitro. Clin Exp Immunol 1975; 20: 273-2 85. [30]D'Ambrosia A, Segoloni G, et al. The modulatory effect of diltiazem on human in vitro alloreactivity when used alone or in combination with cyclosporine A and/or methylprednisolone. Transpl Int 1997; 10:426-431. [31] Wey S P, Wu H Y, et al. Methamphetamine and diazepam suppress antigen-specific cytokine expression and antibody production in ovalbumin-sensitized BALB/c mice. Topical Lett 2008; 181: 157-162. [32]Paul F, Waiczies S, et al. Oral high-dose atorvastatin treatment in relapsing-remitting multiple sclerosis. 2008; PLoS One 3(4): e1928. [33] Delayre-Orthez C, Beck er J, et al. Suppression of allergen-induced airway inflammation and immune response by the peroxisome proliferator-activated receptor-alpha agonist fenofibrate. Eur J Pharmacol 2008; 581: 177-184. [34]Kobashigawa J A, Laks H, et al. The University of California at Los Angeles experience in heart transplantation. Clin. Transpl. 1995; 1995: 129-135. [35] Schmidt W M, Spiel A O, et al. In-vivo effects of simvastatin and rosuvastatin on global gene expression in peripheral blood leucocytes in a human inflammation model. Pharmacogenet Genomics 2008; 18: 109-120. [36]Kim Y C, Kim K K, et al. Simvastatin induces Fox p3+T-regulatory cells by modulation of transforming growth factor-beta signal transduction. Immunology 2010; 130: 484-193. [37] Devoino L, Idova G, et al. Brain neuromediator systems in the immune response control: pharmacological analysis of pre- and postsynaptic mechanisms. Brain Res. 1994; 633: 267-274. [38] Vollmar P, Nessler S, et al. The antidepressant venlafaxine ameliorates murine experimental autoimmune encephalomyelitis by suppression of pro-inflammatory cytokines. Int J Neuropsychopharmacol 2009; 12: 525-536. [39] Taler M, Bar M, et al. Evidence for an inhibitory immunomodulatory effect of selected antidepressants on rat splenocytes: possible relevance to depression and hyperactive-immune disorders. Int Immunopharmacol 2008; 8: 526-533. [40] Pacifici R, Pichini S, et al. Evidence for an inhibitory immunomodulatory effect of selected antidepressants on rat splenocytes: possible relevance to depression and hyperactive-immune disorders. J Pharmacol Exp Ther 2004; 309: 285-292. [41]Pellegrino T C, Bayer B M. Role of central 5-HT (2) receptors in fluoxetine-Induced decreases in T lymphocyte activity. Brain Behav Immun 2002; 16: 87-103. [42]Maes M, Bosmans E, et al. Interleukin-2 and interleukin-6 in schizophrenia and mania: effects of neuroleptics and mood stabilizers. J Psychiatr Res 1995; 29: 141-152. [43]Maes M, Lin A, et al. Negative immunoregulatory effects of noradrenalin through alpha2-adrenoceptor activation. Neuro Endocrinol Lett 2000; 21: 375-382. [44] Mardiney M R Jr., Bredt A B. The immunosuppressive effect of amantadine upon the response of lymphocytes to specific antigens in vitro. Transplantation 1971; 12: 183-188. [45] Kubera M, Basta-Kaim, et al. Effect of repeated amitriptyline administration to mice on the T lymphocyte proliferative activity and natural killer cell cytotoxicity. Pol. J. Phar macol., 1995; 47: 321-326. [46] Yoshiyama Y. Neurodegeneration and inflammation: analysis of a FTDP-17 model mouse. Rinso Shinkeigaku 2008; 48: 910-912. [47] Ashton-Chess J, Meurette G, et al. The study of mitoxantrone as a potential immunosuppressor in transgenic pig renal xenotransplantation in baboons: comparison with cyclophosphamide. Xenotranspla ntation 2004; 11: 112-122. [48] Yao C, Zhang J, Wang L, Guo Y, Tian Z. Inhibitory effects of thyroxin on cytokine production by T cells in mice. Int. Immunopharmacol. 2007; 7: 1747-1754. [49] Kurohara M, Yasuda H, et al. Low-dose warfarin functions as an immunomodulator to prevent cyclophosphamide-induced NOD diabetes. Kobe J. Med. Sci. 2008; 54: E1-E13. [50]Thomas P T, House R V, Bhargava H N. Direct cellular imlmunomodulation produced by diacetylmorphine (heroin) or methadone. Gen. Pharmacol. 1995; 26:123-130. [51]Vallejo R, de Leon-Casasola O, Benyamin R Opioid therapy and immunosuppression: a review. Am. J. Ther. 2004; 11: 354-365. [52] Manchikanti L, Manchikanti K N, et al. Prevalence of side effects of prolonged low or moderate dose opioid therapy with concomitant benzodiazepine and/or antidepressant therapy in chronic non-cancer pain. Pain Physician 2009; 12: 259-267. [53] Cross A H, Waubant E. MS and the B cell Controversy. Biochim Biophys Acta. 2011; 1812(2):231-8. [54] Kappos L, Li D, Calabresi P A, O'Connor P, Bar-Or A, Barkhof F, Yin M, Leppert D, Glanzman R, Tinbergen J, Hauser S L. Ocrelizumab in relapsing-remitting multiple sclerosis: a phase 2, randomised, placebo-controlled, multicentre trial. Lancet. 2011; 378:1779-87. [55]Holmen C, Piehl F, Hillert J, Fogdell-Hahn A, Lundkvist M, Karlberg E, Nilsson P, Dahle C, Feltelius N, Svenningsson A, Lycke J, Olsson T. A Swedish national post-marketing surveillance study of natalizumab treatment in multiple sclerosis. Mult Scler. 2011; 17(6):708-19.[56]Leonard T W, Lynch J, et al. Promoting absorption of drugs in humans using medium-chain fatty acid-based solid dosage forms: GIPET. Expert Opin Drug Deliv 2006; 3: 685-692. [57]Polnok A, Verhoef J C, et al. In vitro evaluation of intestinal absorption of desmopressin using drug-delivery systems based on superporous hydrogels. Int J Pharm 2004; 269: 303-310. [58]van der Merwe S M. Verhoef J C, et al. Trimethylated chitosan as polymeric absorption enhancer for improved per oral delivery of peptide drugs. Eur J Pharm Biopharm 2004; 58: 225-235. [59] Calenoff, E. Interplaying Factors That Effect Multiple Sclerosis Causation and Sustenance. ISRN Neurology. 2012, Article ID 851541, 27 pages. [60] Tsai, M., Grimbaldeston, M., Galli, S. J. Mast cells and immunoregulation/immunomodulation. Adv Exp Med. Biol. 2011; 716:186-211. [61]Kerr M A. The Structure and Function of Human IgA. Biochem J. 1990; 271: 285-296. [62]Johansson S G O. ImmunoCAP Specific IgE Test: An Objective Tool for Research and Routine Allergy Diagnosis. Expert Rev Mol. Diagn. 2004; 4: 273-9. [63] Levy J, Barnett E V, MacDonald N S, Klinenberg J R. Altered immunoglobulin metabolism in systemic lupus Erythematosu s and rheumatoid arthritis. J. Clin Invest. 1970; 49: 708-715. [64] Morell A, Skavaril F, Roseda G, Barandun S. Metabolic Properties of Human IgA Subclasses. Clin exp Immunol. 1973; 13: 521-528. [65] Hellman L/ (2007). Regulation of IgE homeostasis, and the identification of potential targets for therapeutic intervention. Biomed Pharmacother. 2007; 61: 34-49. [66]Esposito P, Barbero L, Caccia P, Caliceti P, D'Antonio et al. PEGylation of growth hormone-releasing hormone (GRF) analogues. Adv Drug Deliv Rev. 2003; 55:1279-91.

### Peptide Sequences

(SEQ ID NO: 1) Artificial Sequence Description of Artificial Sequence Synthetic peptide 2Ala Asp Ala Arg Met1 (SEQ ID NO: 2) Artificial Sequence Description of Artificial Sequence Synthetic peptide 2 His Ser Tyr Gln Glu1 (SEQ ID NO: 3) Artificial Sequence Description of Artificial Sequence Synthetic peptide 3 Asp His Ser Tyr Gln Glu1 (SEQ ID NO: 4) Artificial Sequence Description of Artificial Sequence Synthetic peptide 4 Arg Asn Val Arg Phe1 (SEQ ID NO: 5) Artificial Sequence Description of Artificial Sequence Synthetic peptide 5 Val Thr Leu Arg Ile1 (SEQ ID NO: 6) Artificial Sequence Description of Artificial Sequence Synthetic peptide 6 Ile Glu Asn Leu His1 (SEQ ID NO: 7) Artificial Sequence Description of Artificial Sequence Synthetic peptide 7 Asn Leu His Arg Thr Phe Glu1 (SEQ ID NO: 8) Artificial Sequence Description of Artificial Sequence Synthetic peptide 8 Asn Leu His Arg Thr 1 (SEQ ID NO: 9) Artificial Sequence Description of Artificial Sequence Synthetic peptide 9 Leu His. Arg Thr Phe1 (SEQ ID NO: 10) Artificial Sequence Description of Artificial Sequence Synthetic peptide 10 His Arg Thr Phe Glu1 (SEQ ID NO: 11) Artificial Sequence Description of Artificial Sequence Synthetic peptide 11 Lys Gly Thr Gln Phe1 (SEQ ID NO: 12) Artificial Sequence Description of Artificial Sequence Synthetic peptide 12 Asp Asn Glu Val Phe Gly Glu Ala1 (SEQ ID NO: 13) Artificial Sequence Description of Artificial Sequence Synthetic peptide 13 Asp Asn Glu Val Phe1 (SEQ ID NO: 14) Artificial Sequence Description of Artificial Sequence Synthetic peptide 14 Asn Glu Val Phe Gly1 (SEQ ID NO: 15) Artificial Sequence Description of Artificial Sequence Synthetic peptide 15 Glu Val Phe Gly Glu1 (SEQ ID NO: 16) Artificial Sequence Description of Artificial Sequence Synthetic peptide 16 Val Phe Gly Glu Ala1 (SEQ ID NO: 17) Artificial Sequence Description of Artificial Sequence Synthetic peptide 17 Gln Asp Thr Ala Val Thr1 (SEQ ID NO: 18) Artificial Sequence Description of Artificial Sequence Synthetic peptide 18 Gln Asp Thr Ala Val1 (SEQ ID NO: 19) Artificial Sequence Description of Artificial Sequence Synthetic peptide 19 Asp Thr Ala Val Thr1 (SEQ ID NO: 20) Artificial Sequence Description of Artificial Sequence Synthetic peptide 20 Pro Lys Asn Ala Trp1 (SEQ ID NO: 21) Artificial Sequence Description of Artificial Sequence Synthetic peptide 21 Asp Asn Thr Phe Lys Asp1 (SEQ ID NO: 22) Artificial Sequence Description of Artificial Sequence Synthetic peptide 22 Asp Asn Thr Phe Lys1 (SEQ ID NO: 23) Artificial Sequence Description of Artificial Sequence Synthetic peptide 23 Asn Thr Phe Lys Asp1 (SEQ ID NO: 24) Artificial Sequence Description of Artificial Sequence Synthetic peptide 24 Leu Gln Thr Ile Gln Glu1 (SEQ ID NO: 25) Artificial Sequence Description of Artificial Sequence Synthetic peptide 25 Leu Gln Thr Ile Gln1 (SEQ ID NO: 26) Artificial Sequence Description of Artificial Sequence Synthetic peptide 26 Gln Thr Ile Gln Glu1 (SEQ ID NO: 27) Artificial Sequence Description of Artificial Sequence Synthetic peptide 27 Lys Asp Ser His His Pro Ala1 (SEQ ID NO: 28) Artificial Sequence Description of Artificial Sequence Synthetic peptide 28 His Gly Arg Thr Gln1 (SEQ ID NO: 29) Artificial Sequence Description of Artificial Sequence Synthetic peptide 29 Tyr Lys Asp Ser His His Pro Ala1 (SEQ ID NO: 30) Artificial Sequence Description of Artificial Sequence Synthetic peptide 30 Tyr Lys Asp Ser His1 (SEQ ID NO: 31) Artificial Sequence Description of Artificial Sequence Synthetic peptide 31 Lys Asp Ser His His1 (SEQ ID NO: 32) Artificial

TABLE 1
Estimation of Average Amino Acid Diameter

| Molar | | Amino | Nanometer | Ångströms |
|---|---|---|---|---|
| Mass | | Acids: | Diameter: | Diameters |
| 89.1 | A | Alanine | 0.69 ** | 6.9 |
| 132.1 | N | Asparagine | 1.02 | 10.2 |
| 133.1 | D | Aspartic acid | 1.03 | 10.3 |
| 121.6 | C | Cysteine | 0.94 | 9.4 |
| 147.1 | E | Glutamic acid | 1.14 | 11.4 |
| 146.1 | Q | Glutamine | 1.13 | 11.3 |
| 75.1 | G | Glycine | 0.58 | 5.8 |
| 115.1 | P | Proline | 0.89 | 8.9 |
| 105.1 | S | Serine | 0.81 | 8.1 |
| 181.2 | Y | Tyrosine | 1.40 | 14.0 |
| 174.2 | R | Arginine | 1.35 | 13.5 |
| 155.2 | H | Histidine | 1.20 | 12.0 |
| 131.2 | I | Isoleucine | 1.02 | 10.2 |
| 131.2 | L | Leucine | 1.02 | 10.2 |
| 146.2 | K | Lysine | 1.13 | 11.3 |
| 149.2 | M | Methionine | 1.16 | 11.6 |
| 165.2 | F | Phenylalanine | 1.28 | 12.8 |
| 119.1 | T | Threonine | 0.92 | 9.2 |
| 204.2 | W | Tryptophan | 1.58 | 15.8 |
| 117.5 | V | Valine | 0.91 | 9.1 |

| | |
|---|---|
| Average Ångströms Diameter per Amino Acid => | 10.6 |

** H. Hasizumi, Yamagishi, et. al, Clay Minerals, 37, 551(2002).

TABLE 2
Microgram of Mimotopic Peptide Construct per Milliliter of Coating
Buffer Applied to MS Microplate Test Wells (100 μL Solution/well)

| | Amino Acid | | |
|---|---|---|---|
| | Sequence of | | |
| | amino-ADOOA- | μg/mL | Construct |
| | ADOOA Peptide | of Peptide | Molar Mass |
| | Construct | Construct | (kilodaltons) |
| 1 | ADARM | 0.26 | 0.56 |
| 2 | HSYQE | 0.31 | 0.66 |
| 3 | RNVRF | 0.33 | 0.69 |
| 4 | VTLRI | 0.28 | 0.6 |
| 5 | IENLH | 0.29 | 0.62 |
| 6 | NLHRTFE | 0.5 | 1.06 |
| 7 | KTGQF | 0.27 | 0.58 |
| 8 | DNTFKD | 0.39 | 0.82 |
| 9 | HGRTQ | 0.28 | 0.6 |
| 10 | LQTIQE | 0.34 | 0.73 |
| 11 | PKNAW | 0.34 | 0.73 |
| 12 | QDTAVT | 0.3 | 0.63 |
| 13 | YKDSHHPA | 0.45 | 0.95 |
| 14 | DNEVFGEA | 0.5 | 0.9 |

TABLE 3a

| Anti-Inflammatory Agent | | | Calenoff |
|---|---|---|---|
| (I.D. Numbers Listed on | | | 2012 Article 1 |
| bottom of Individual Plots: | | Immunosuppressive Effects: | Reference List: |
| (1) | Mesalazine | Potent and Specific Inhibitor | 25. |
| | | of Nuclear Factor kappa B. | |
| Anti-Convulsants: | | | |
| (2) | Dilantin(Phenytoin sodium) | Humoral Immune Suppressant | 26. |
| (3) | Zonisamide | Suppression of IFN-gamma | 27. |
| | | Production by Lymphocytes. | |
| Atypical Antipsychotics: | | | |
| (4) | Olanzapine(Zyprexa, etc) | Suppress Tumor Necrosis Factor, | 28. |
| | | (TNF)-alpha, Interleukin | |
| | | (IL)-6, and Up-regulates IL-10 | |
| Benzodiazepines: | | | |
| (5) | Alprazolam(Xanax) | Inhibits proliferative responses | 29. |
| | | of both B- and T-cells | |
| (6) | Clonazepam | Depression of Cellular and | 30. |
| | | Humoral ImmuneResponse. | |
| (7) | Diltiazem | Induces Direct Immunosuppression. | 31. |

TABLE 3b

| Anti-Inflammatory Agent | | | Calenoff |
|---|---|---|---|
| (I.D. Numbers Listed on | | | 2012 Article 1 |
| bottom of Individual Plots: | | Immunosuppressive Effects: | Reference List: |
| (8) | Diazepam (Valium) | Markedly Suppresses Antigen- | 32. |
| | | specific Antibody Production | |
| | | and T-cell Reactivity. | |
| Cholesterol Lowering Drugs: | | | |
| (9) | Atorvastatin (Lipitor) | Increases in IL-10 Production. | 33. |
| | | IL-10 Mediates Immune Suppression. | |
| (10) | Fenofibrate | A Peroxisome Proliferator- | 34. |
| | (Reduces lipoproteins) | Activated Receptor alpha | |
| | | Agonist. | |
| (11) | Pravastatin | B. Lymphocyte and T. | 35. |
| | | Lymphocyte Suppression. | |
| (12) | Rosuvastatin (Crestor) | Post-Transcriptional Level | 36. |
| | | of Genetic Expression of | |
| | | Inflammatory Process. | |
| (13) | Simvastatin (Zocor) | Mediates Induction of Foxp3 (+) | 37. |
| | | T Cells Which Mediate | |
| | | Immunosuppression. | |
| Dopamine Reuptake Inhibitors | | | |
| (antidepressants): | | | |
| (14) | Bupropion (Wellbutrin, etc.) | Involved in Inhibiting | 38. |
| | | Neuro-immuno0modulation. | |

TABLE 3c

| Anti-Inflammatory Agent | | | Calenoff |
|---|---|---|---|
| (I.D. Numbers Listed on | | | 2012 Article 1 |
| bottom of Individual Plots: | | Immunosuppressive Effects: | Reference List: |
| Serotonin-norepinephrine Re- | | | |
| uptake Inhibitors (SNRI anti- | | | |
| Depressants): | | | |
| (15) | Venlafaxine | Suppresses pro-Inflammatory | 39. |
| | | Cytokines | |
| Selective Serotonin | | | |
| Rentidepressants): | | | |
| (16) | Paroxetine (Trade names: | Inhibit Splenocyte Viability. | 40. |
| | Seroxat, Paxil) | Decreases CD4 T-Helper Cells. | 41. |
| (17) | Fluoxethine (Prozac) | Decreases T Lymphocyte | 42. |
| | | Activity. | |
| (18) | Sertraline hydrochloride | Suppression of Antigen- | 43. |
| | (Zoloft) | specific T(H)1 Responses. | |
| | | Inhibition of Interferon | |
| | | gamma and Stimulation of | |
| | | Interleukin-10. | |
| (19) | Clomipramine | As per Sertaline. | 44. |
| (20) | Trazodone (Desryl, | As per Sertaline. | 44. |
| | Oleptro, Beneficat, | | |
| | Deprax, Desirel, | | |
| | Molipaxin, Thombran, | | |
| | Trazorel, Trialodine, | | |
| | Trittico, Mesyrel). | | |

TABLE 3d

| Anti-Inflammatory Agent | | | Calenoff |
|---|---|---|---|
| (I.D. Numbers Listed on | | | 2012 Article 1 |
| bottom of Individual Plots: | | Immunosuppressive Effects: | Reference List: |
| Other Immunosuppresants: | | | |
| (21) | Amantadine | Inhibits Antigen-specific T | 45. |
| | | and NK Cell Responses. | |
| (22) | Amitriptyline (Elavil, | Decrease in the | 46. |
| | Tryptizol, Laroxyl, | Proliferation of | |
| | Sarotex) | Splenocytes and in NK | |
| | | Activity. | |
| (23) | Clonidine (a direct- | Stimulates Production of | 47. |
| | acting α2 adrenergic | IL-10 (an anti-Inflammatory | |
| | agonist). | Cytokine that Reduces Serum | |
| | | Antibody Production.) | |
| (24) | Depakote (Valproate | Suppresses IL-6 and/or | 43. |
| | semi-sodium used to | IL-6R-related Mechanisms. | |
| | treat major depressive | | |
| | disorder.) | | |
| (25) | Donepezil (Aricept) | Reverseable Acetyl cholinesterase | 48. |
| | | Inhibitor. Suppresses Neuroinflam- | |
| | | ation of the Brain. | |
| (26) | Mitoxantrone (Novantrone) | Chemotherapeutic | 49. |
| | | Agent. Depletes B cells. | |
| (27) | Levoxyl (Levothyroxine, | Inhibits Cytokine | 50. |
| | Synthroid. | Production in T Cells. | |

TABLE 3e

| Anti-Inflammatory Agent | | | Calenoff |
|---|---|---|---|
| (I.D. Numbers Listed on | | | 2012 Article 1 |
| bottom of Individual Plots: | | Immunosuppressive Effects: | Reference List: |
| (28) | Warfarin (Coumadin) | Suppresses IL-6 secretion. | 51. |
| | | Serves as immunosuppressant. | |
| (29) | Heroin and Methadone. | Suppression of Cellular and | 52. |
| | | Humoral Immunity. | |
| (30) | Morphine. | Suppression of Cellular and | 52. |
| | | Humoral Immunity. | |
| (31) | Oxycodone & Propoxyphene | Suppression of Cellular and | 52. |
| | | Humoral Immunity. | |
| (32) | Prednisone | Catabolic Steroid. | |
| | | Suppression of Cellular and | |
| | | Humoral Immunity. | |

TABLE 4a
MS-Specific Peptides for Diagnosis and Therapy

| | | | | | Single- | |
|---|---|---|---|---|---|---|
| | | | Full-length | | epitope, | |
| | Myelin | | Mimotopic | | Pentameric | |
| | Protein | | Peptides | H.I. | Equivalents | H.I. |
| 1 | PLP | myelin surface | ADARM | 3.7 | ADARM | 3.7 |
| 2 | MOG | myelin surface | HSYQE | 0.7 | HSYQE | 0.7 |
| 3 | | ″ | RNVRF | 2 | RNVRF | 2.2 |
| 4 | | ″ | VTLRI | −2.5 | VTLRI | −2.5 |
| 5 | MOG | myelin subsurface | IENLH | −0.9 | IENLH | −0.9 |
| 6 | | ″ | NLHRTFE | 1 | NLHRT | 0.5 |
| 7 | | ″ | | | LHRTF | −2.2 |
| 8 | | ″ | | | HRTFE | 2.6 |
| 9 | | ″ | KGTQF | 0.7 | KGTQF | 0.7 |
| | MBP | myelin subsurface | | | | |
| 10 | | Isoforms-1,2 | DNEVFGEA | 5 | DNEVF | 2.2 |
| 11 | | | | | NEVFG | 2.0 |
| 12 | | | | | EVFGE | 2.0 |
| 13 | | | | | VFGEA | −1.5 |
| 14 | | Isoforms-1,2 | QDTAVT | 0.4 | QDTAV | 0.8 |
| 15 | | | | | DTAVT | 0.2 |
| 16 | | Isoforms-1,2 | PKNAW | −1.0 | PKNAW | −1.0 |
| 17 | | | DNTFKD | 6.3 | DNTFK | 3.3 |
| 18 | | | | | NTFKD | 3.3 |
| 19 | | Isoform-2 | LQTIQE | −1.0 | LQTIQ | −3.6 |
| 20 | | | | | QTIQE | 1.2 |
| | | Isoform-1 | KDSHHPA | 3 | KDSHH | 5.3 |
| | | | | | DSHHP | 2.3 |
| | | | | | SHHPA | −1.2 |
| 21 | | Isoform-3 | HGRTQ | 2 | HGRTQ | 2.3 |
| 22 | | | YKDSHHPA | 3 | YKDSH | 2.5 |
| 23 | | | | | KDSHH | 5.3 |
| 24 | | | | | DSHHP | 2.3 |

| | | | |
|---|---|---|---|
| 25 | Bold, Large Font = Cornerstone Epitopes. | SHHPA | −1.2 |

TABLE 4b

| | Peptide | Linker,Amino Acids' Molar Mass | | | | | | Peptide | Peptide | Free | Free |
|---|---|---|---|---|---|---|---|---|---|---|---|
| | AminoAcid | ADOOA | | | | | | Construct | Construct | Peptide | Peptide |
| | Sequence | Linker | aa1 | aa2 | aa3 | aa4 | aa5 | Molar Mass | HI | Molar Mass | HI |
| 1 | ADARM | 771 | 89 | 133 | 89 | 174 | 131 | 1387 | >3 | 617 | 3.7 |
| 2 | HSYQE | 771 | 155 | 105 | 181 | 146 | 147 | 1506 | >3 | 735 | 0.7 |
| 3 | RNVRF | 771 | 174 | 132 | 117 | 174 | 165 | 1534 | >3 | 763 | 2.2 |
| 4 | VTLRI | 771 | 117 | 119 | 131 | 174 | 131 | 1443 | >3 | 672 | −2.5 |
| 5 | IENLH | 771 | 131 | 147 | 132 | 131 | 155 | 1143 | >3 | 696 | −0.9 |
| 6 | NLHRT | 771 | 132 | 131 | 155 | 174 | 119 | 1188 | >3 | 711 | 0.5 |
| 7 | LHRTF | 771 | 131 | 155 | 174 | 119 | 165 | 1054 | >3 | 745 | −2.2 |
| 8 | HRTFE | 771 | 155 | 174 | 119 | 165 | 147 | 1532 | >3 | 761 | 2.6 |
| 9 | KTGQF | 771 | 146 | 119 | 75 | 146 | 165 | 1110 | >3 | 652 | 0.3 |
| 10 | DNEVF | 771 | 133 | 132 | 147 | 117 | 165 | 1466 | >3 | 695 | 2.2 |
| 11 | NEVFG | 771 | 132 | 147 | 117 | 165 | 75 | 1408 | >3 | 637 | 2.0 |
| 12 | EVFGE | 771 | 147 | 117 | 165 | 75 | 147 | 1423 | >3 | 652 | 2.0 |
| 13 | VFGEA | 771 | 117 | 165 | 75 | 147 | 89 | 1100 | >3 | 594 | −1.5 |
| 14 | QDTAV | 771 | 146 | 133 | 119 | 89 | 117 | 1376 | >3 | 605 | 0.8 |
| 15 | DTAVT | 771 | 133 | 119 | 89 | 117 | 119 | 1348 | >3 | 578 | 0.2 |
| 16 | PKNAW | 771 | 115 | 146 | 132 | 89 | 204 | 1458 | >3 | 687 | −1.0 |
| 17 | DNTFK | 771 | 133 | 132 | 119 | 165 | 146 | 1467 | >3 | 696 | 3.3 |
| 18 | NTFKD | 771 | 132 | 119 | 165 | 146 | 133 | 1467 | >3 | 696 | 3.3 |
| 19 | LQTIQ | 771 | 131 | 146 | 119 | 131 | 146 | 1186 | >3 | 674 | −3.6 |
| 20 | QTIQE | 771 | 146 | 119 | 131 | 146 | 147 | 1461 | >3 | 690 | 1.2 |
| 21 | HGRTQ | 771 | 155 | 75 | 174 | 119 | 146 | 1441 | >3 | 670 | 2.3 |
| 22 | YKDSH | 771 | 181 | 146 | 133 | 105 | 155 | 1492 | >3 | 721 | 2.5 |
| 23 | KDSHH | 771 | 146 | 133 | 105 | 155 | 155 | 1466 | >3 | 695 | 5.3 |
| 24 | DSHHP | 771 | 133 | 105 | 155 | 155 | 115 | 1143 | >3 | 664 | 2.3 |
| 25 | SHHPA | 771 | 105 | 155 | 155 | 115 | 89 | 1188 | >3 | 620 | −1.2 |

ADOOA-ADOOA = Hydrophobic Peptide Linker
aa: amino acid
ADOOA-ADOOA = (Fmoc-8-Amino-3,6-Dioxaoctanoic Acid-Fmoc-8-Amino-3,6-Dioxaoctanoic Acid)₂
Linker Molar Mass = 385.4 × 2 = 771
HI: Hydrophobic Index

TABLE 5
a. Baseline Serum Antibody Levels

| | mg/mL | | gm/mL | Refer- | Half-life | Refer- |
|---|---|---|---|---|---|---|
| | serum | | serum | ence | (days) | ence |
| IgA | 3.3 | | 0.00328 | 61 | 5.9 | 64 |
| IgE | 1.47 × 10⁻¹⁰ | * | 1.47 × 10⁻⁷ | 62 | 2.5 | 65 |
| IgG | 12.5 | | 0.0125 | 63 | 21 | 65 |
| IgM | 1.0 | | 0.001 | 63 | 9.3 | 63 |

* 60 International Units IgE/mL serum: 150 ng/mL (highest quantity possible)

TABLE 6
mole = weight of sample (in grams)/relative molar mass

| | (b) | | (c) | (d) | | | (e) |
|---|---|---|---|---|---|---|---|
| | gm/mL | Molar | Mole/mL | Valence | Mole/mL Serum | | Valence Mole per |
| | serum | Mass | serum | No. | times valence number | | 2,750 mL of serum |
| IgA | 0.00328 | 160,000 | 0.000000020500000000 | 2 | 0.000000041000000000 | | 0.000112750000000000 |
| IgE | 0.000000001479 | 188,000 | 0.000000000000007867 | 2 | 0.000000000000015734 | | 0.000000000043268617 |
| IgG | 0.0125 | 150,000 | 0.000000083333333333 | 2 | 0.000000166666666667 | | 0.000458333333333333 |
| IgM | 0.001 | 900,000 | 0.000000001111111111 | 10 | 0.000000011111111111 | | 0.000030555555555556 |
| | | | Sum of mole values | = | 0.000000218777793512 | = | 0.000601638932157506 |

TABLE 7

| | | (f) |
|---|---|---|
| | | If Only 1 in 1,000,000 of antibody |
| | (e) | isotypes was epitope-specific, |
| | Valence Mole per | It would comprise following |
| | 2,750 mL of serum | specific valence mole: |
| IgA | 0.00011275 | 0.00000000011275 |
| IgE | 0.000000000043 | 0.00000000000000 |
| IgG | 0.00045833 | 0.00000000045833 |
| IgM | 0.00003056 | 0.00000000003056 |
| Total: | 0.00060163893216 | 0.00000000060164 |

TABLE 8

| | Cornerstone, | | Functional | |
|---|---|---|---|---|
| (g) | dimer-blocking peptides | mm | Molar Mass | HI |
| MS | ADARM | 543.7 | 543.7 | 3.7 |
| MS | DHSYQE | 734.7 | 758.8 | 3.7 |
| IC | KTGQF₂ | 561.07 | 1122.1 | 0.3 |
| | | | 2424.64 | |

mole = weight of sample (in grams)/relative molar mass
0.0060164 = weight of sample (in grams)/molar mass

| | | |
|---|---|---|
| (h) | 0.0060164 * 2424.64 = | :weight of Epitope neutralizing Peptides |
| | | sample (in grams) |
| (i) | 14.5876041 | grams |
| (j) | 14.6 | micrograms of 3 peptide mix is |
| | | required per day to neutralize |
| | | all single epitope-specific, |
| | | in-vivo antibodies. |

10 mg/mL Therapeutic Solution Formulation:

Polypeptide Laboratories, San Diego, Ca USA

| | | | |
|---|---|---|---|
| 99% pure | ADARM | 484.5 | mg |
| ″ | DHSYQE | 676.2 | ″ |
| ″ | KTGQF₂ | 1000 | ″ |
| | | 2161 | mg |

| | | | |
|---|---|---|---|
| 50% Glycerine, Product Number | 216.0728608 | mL | 10 mg/mL |
| DG50-100S | | | |

Allergy Laboratories, Inc., Oklahoma City, OK USA

TABLE 9

| | Cornerstone, | | Functional | |
|---|---|---|---|---|
| (g) | dimer-blocking peptides | mm | Molar Mass | HI |
| MS | DNTFKD | 828.8 | 828.8 | 6.3 |
| IC | HGRTQ | 669.7 | 1339.4 | 2.3 |
| IC | QDTAVT | 723.7 | 1447.4 | 0.4 |
| | | | 3615.6 | |

mole = weight of sample (in grams)/relative molar mass
0.0060164 = weight of sample (in grams)/molar mass

| | | |
|---|---|---|
| (h) | 0.0060164 * 3615.6 = | :weight of Epitope neutralizing |
| | | Peptides sample (in grams) |
| (i) | 21.8 | grams |
| (j) | 0.0218 | milligram of 3 peptide mix is |
| | | required per day to neutralize |
| | | all single epitope-specific, |
| | | in-vivo antibodies. |

10 mg/mL Therapeutic Solution Formulation:

Polypeptide Laboratories, San Diego, Ca USA

| | | | | |
|---|---|---|---|---|
| 99% pure | DNTFKD | 1000.0 | 828.8 | mg |
| ″ | HGRTQ | 669.7 | 1339.4 | ″ |
| ″ | QDTAVT | 723.7 | 1447.4 | ″ |
| | | | 3615.6 | mg |

| | | | |
|---|---|---|---|
| 50% Glycerine, Product Number DG50-100S | 275 | mL | per mL: |

| | |
|---|---|
| Allergy Laboratories, Inc., Oklahoma City, OK USA | 13.148 mg |

TABLE 10
Assumes 10 minute intravascular half-life
due to renal clearance and enzymatic degradation
of ADARM, DHSYQE, & KTGQF peptides:

| | 40 mg (4 mL) | | | | |
|---|---|---|---|---|---|
| | of 3 Peptide | | | | |
| Dilu- | Mix (40,000 | | | | |
| tion: | μg) S.Q. | minutes | Hours | Days | |
| | 40,000.00 | | | | |
| ½ | 20,000.00 | 10 | 0 | 0 | |
| ¼ | 10,000.00 | 20 | 0 | 0 | |
| ⅛ | 5,000.00 | 40 | 1 | 0 | |
| 1/16 | 2,500.00 | 80 | 1 | 0 | |
| 1/32 | 1,250.00 | 160 | 3 | 0 | |
| 1/64 | 625.00 | 320 | 5 | 0 | |
| 1/128 | 312.50 | 640 | 11 | 0 | |
| 1/256 | 156.25 | 1,280 | 21 | 1 | |
| 1/512 | 78.13 | 2,560 | 43 | 2 | |
| 1/1,024 | 39.06 | 5,120 | 85 | 4 | |
| 1/2,048 | 19.53 | 10,240 | 171 | 7 | 14.6 μg |
| 1/4,096 | 9.77 | 20,480 | 341 | 14 | (empiri- |
| 1/8,192 | 4.88 | 40,960 | 683 | 28 | cally |
| 1/16,384 | 2.44 | 81,920 | 1,365 | 57 | titered |
| | 1.22 | 163,840 | 2,731 | 114 | to attain |
| | 0.61 | 327,680 | 5,461 | 228 | zero serum |
| | 0.31 | 655,360 | 10,923 | 455 | IgE level) |
| | 0.15 | 1,310,720 | 21,845 | 910 | |
| | 0.08 | 2,621,440 | 43,691 | 1,820 | |
| | 0.04 | 5,242,880 | 87,381 | 3,641 | |

3 Peptide Mix: 0.0168 mg/day, (14.6 μg/day)

TABLE 11
Assumes 10 minute intravascular half-life
due to renal clearance and enzymatic degradation
of DNTFKD, HGRTQ, & QDTAVT peptides:

| | 52.6 mg (4 mL) | | | | |
|---|---|---|---|---|---|
| | of 3 Peptide | | | | |
| Dilu- | Mix (52,600 | | | | |
| tion: | μg) S.Q. | minutes | Hours | Days | |
| | 52,600.00 | | | | |
| ½ | 26,300.00 | 10 | 0 | 0 | |
| ¼ | 13,150.00 | 20 | 0 | 0 | |
| ⅛ | 6,575.00 | 40 | 1 | 0 | |
| 1/16 | 3,287.50 | 80 | 1 | 0 | |
| 1/32 | 1,643.75 | 160 | 3 | 0 | |
| 1/64 | 821.88 | 320 | 5 | 0 | |
| 1/128 | 410.94 | 640 | 11 | 0 | |
| 1/256 | 205.47 | 1,280 | 21 | 1 | |
| 1/512 | 102.73 | 2,560 | 43 | 2 | |
| 1/1,024 | 51.37 | 5,120 | 85 | 4 | |
| 1/2,048 | 25.68 | 10,240 | 171 | 7 | 21.8 μg |
| 1/4,096 | 12.84 | 20,480 | 341 | 14 | (1-2 |
| 1/8,192 | 6.42 | 40,960 | 683 | 28 | weeks |
| 1/16,384 | 3.21 | 81,920 | 1,365 | 57 | Rx range) |
| | 1.61 | 163,840 | 2,731 | 114 | |
| | 0.80 | 327,680 | 5,461 | 228 | |
| | 0.40 | 655,360 | 10,923 | 455 | |
| | 0.20 | 1,310,720 | 21,845 | 910 | |
| | 0.10 | 2,621,440 | 43,691 | 1,820 | |
| | 0.05 | 5,242,880 | 87,381 | 3,641 | |

3 Peptide Mix: 0.0218/mg/day, (21.8 μg/day)

TABLE 12A
3 Minutes Substrate Incubtion, Ascent Software: Measurement count: 1 Filter: 0 Scaling Factor: 4

| | ADARM | ADARM | HSYQE | HSYQE | Blank | ADARM | ADARM | HSYQE | HSYQE | Blank | |
|---|---|---|---|---|---|---|---|---|---|---|---|
| 1 | 2 | 3 | 4 | 5 | 6 | 7 | 8 | 9 | 10 | 11 | 12 |
| A | 852 | 503 | 744 | 389 | 474 | 5,834 | 3,016 | 6,160 | 3,549 | 17,900 | |
| B | 655 | 544 | 558 | 496 | 496 | 6,223 | 2,754 | 7,599 | 3,178 | 3,138 | |
| C | 667 | 504 | 528 | 514 | 521 | 6,921 | 3,098 | 6,229 | 3,633 | 2,897 | |
| D | 867 | 651 | 1,038 | 605 | 697 | 6,436 | 4,179 | 7,368 | 4,458 | 4,272 | |
| E | 1,541 | 1,145 | 1,393 | 1,552 | 1,391 | 5,748 | 2,432 | 4,621 | 2,565 | 1,883 | |
| F | 952 | 662 | 704 | 700 | 719 | 8,209 | 3,702 | 6,401 | 6,678 | 3,209 | |
| G | 915 | 433 | 520 | 438 | 450 | 6,295 | 2,312 | 6,593 | 3,302 | 2,695 | |
| H | 803 | 407 | 682 | 500 | 435 | 6,049 | 3,756 | 6,513 | 3,539 | 3,062 | |
| A | 852 | 503 | 744 | | 474 | 5,834 | 3,016 | 6,160 | 3,549 | | |
| B | 655 | 544 | 558 | 496 | 496 | 6,223 | 2,754 | | 3,178 | 3,138 | |
| C | | 504 | 528 | 514 | 521 | 6,921 | 3,098 | 6,229 | 3,633 | 2,897 | |
| D | 867 | 651 | 1,038 | 605 | 697 | 6,436 | | 7,368 | 4,458 | 4,272 | |
| E | | | | | | | 2,432 | | | | |
| F | 952 | 662 | 704 | 700 | 719 | | 3,702 | 6,401 | | 3,209 | |
| G | 915 | 433 | | 438 | 450 | 6,295 | | 6,593 | 3,302 | 2,695 | |
| H | 803 | | 682 | 500 | | 6,049 | 3,756 | 6,513 | 3,539 | 3,062 | |
| Average | 841 | 549 | 709 | 542 | 560 | 6,293 | 3,126 | 6,544 | 3,610 | 3,436 | |
| SD> | 105 | 90 | 182 | 94 | 118 | 371 | 522 | 436 | 449 | 734 | |
| Avg + 2SD> | 1,050 | 730 | 1,073 | 730 | 795 | 7,036 | 4,170 | 7,416 | 4,509 | 4,904 | |

TABLE 12b
8 Weeks Following MS Therapy Commencement
Formula to Estimate MS Activity Factor (MAF) = Net IgE/Net KL × 1,000,000

| | | ADARM | ADARM | HSYQE | HSYQE | | ADARM | ADARM | HSYQE | HSYQE | | |
|---|---|---|---|---|---|---|---|---|---|---|---|---|
| | | Pre | Post | Pre | Post | | Pre | Post | Pre | Post | | |
| | 1 | 2 | 3 | 4 | 5 | 6 | 7 | 8 | 9 | 10 | 11 | 12 |
| Net | | 254 | −65 | 1,862 | −65 | | 2,857 | −309 | 3,108 | −395 | | |
| Signal> | | | | | | | | | | | | |

| | | | | | |
|---|---|---|---|---|---|
| ADARM before | KL | 71,425,000 | during | KL | −7,725,000 |
| Treatment: | E/KL | 0.0000036 | Treatment: | E/KL | 0.0000084 |
| | ×1 million | 3.6 | | ×1 million | −8.4 |
| HSYQE before | KL | 77,700,000 | during | KL | −9,875,000 |
| Treatment: | E/KL | 0.0000240 | Treatment: | E/KL | 0.00000661 |
| | ×1 million | 24.0 | | ×1 million | −6.6 |

KL = specific (kappa + lambda) signal *25,000 (Compensates for 1/25,000 serum dilution)

1. An isolated peptide homologous to a protein epitope, wherein the peptide has a mimotopic amino acid sequence found on the surface of only one protein transcribed from the human genome and a net hydrophilicity index value of −2.5 to 6.3.

2. A peptide of claim 1 comprising 5 amino acids.

3. The peptide of claim 1 wherein the peptide is selected from or homologous in total or in part to any one of the following amino acid sequences: ADARM (SEQ ID NO: 1), DHSYQE (SEQ ID NO: 3), HSYQE (SEQ ID NO: 2), RNVRF (SEQ ID NO: 4), VTLRI (SEQ ID NO: 5), IENLH (SEQ ID NO: 6), NLHRTFE (SEQ ID NO: 7), KTGQF (SEQ ID NO: 11), DNEVFGEA (SEQ ID NO: 12), QDTAVT (SEQ ID NO: 17), PKNAW (SEQ ID NO: 20), DNTFKD (SEQ ID NO: 21), LQTIQE (SEQ ID NO: 24), YKDSHHPA (SEQ ID NO: 29), and HGRTQ (SEQ ID NO: 28).

4. A dimer composition of a protein comprising two peptides of claim 2, each of which is homologous to a myelin protein epitope, wherein a first epitope is located approximately 40-100 Ångströms from the second epitope.

5. The composition of claim 4, wherein the first and second epitope comprise a pair selected from the following pairs of sequences: (a) ADARM (SEQ ID NO: 1) and ADARM (SEQ ID NO: 1); (b) HSYQE (SEQ ID NO: 3) and VTLRI (SEQ ID NO: 5); (c) RNVRF (SEQ ID NO: 4) and HSYQE (SEQ ID NO: 2); (d) IENLH (SEQ ID NO: 6) and KTGQF (SEQ ID NO: 11); (e) NLHRT (SEQ ID NO: 8) and KTGQF (SEQ ID NO: 11); (f) LHRTF (SEQ ID NO: 9) and KTGQF (SEQ ID NO: 11); (g) HRTFE (SEQ ID NO: 10) and KTGQF (SEQ ID NO: 11); (h) DNEVF (SEQ ID NO: 13) and QDTAV (SEQ ID NO: 18); (i) NEVFG (SEQ ID NO: 14) and QDTAV (SEQ ID NO: 18); (j) EVFGE (SEQ ID NO: 15) and QDTAV (SEQ ID NO: 18); (k) VFGEA (SEQ ID NO: 16) and QDTAV (SEQ ID NO: 18); (1) DNEVF (SEQ ID NO: 13) and DTAVT (SEQ ID NO: 19); (m) NEVFG (SEQ ID NO: 14) and DTAVT (SEQ ID NO: 19); (n) EVFGE (SEQ ID NO: 15) and DTAVT (SEQ ID NO: 19); (o) VFGEA (SEQ ID NO: 16) and DTAVT (SEQ ID NO: 19); (p) QDTAV (SEQ ID NO: 18) and PKNAW (SEQ ID NO: 20); (q) DTAVT (SEQ ID NO: 19) and PKNAW (SEQ ID NO: 20); (r) DNTFK (SEQ ID NO: 22) and LQTIQ (SEQ ID NO: 25); (s) NTFKD (SEQ ID NO: 23) and LQTIQ (SEQ ID NO: 25); (t) DNTFK (SEQ ID NO: 22) and QTIQE (SEQ ID NO: 24); (u) NTFKD (SEQ ID NO: 23) and QTIQE (SEQ ID NO: 26); (v) YKDSH (SEQ ID NO: 30) and HGRTQ (SEQ ID NO: 28); (w) KDSHH (SEQ ID NO: 31) and HGRTQ (SEQ ID NO: 28); (x) DSHHP (SEQ ID NO: 32) and HGRTQ (SEQ ID NO: 28); and (y) SHHPA (SEQ ID NO: 33) and HGRTQ (SEQ ID NO: 28).

6. A peptide construct comprising the peptide of claim 1, wherein the first or last amino acid of the peptide is attached to a hydrophilic linker possessing a distally free amino group or other, similar point of attachment.

7. The peptide constructs of claim 6, wherein the linker is a monomer or polymer of 8-Fmoc-amino-3,6-dioxa-octanoic acid.

8. An immunoassay to determine the amount of IgE antibody specific to an epitope of a protein in a biological fluid sample comprising (a) contacting the sample with at least one peptide of claim 3, (b) determining the amount of IgE antibody bound to the peptide, thereby determining the amount of IgE antibody specific to an epitope of a protein in the sample.

9. An immunoassay to determine the amount of non-IgE antibody specific to an epitope of a protein in a biological fluid sample comprising (a) contacting the sample with at least one peptide of claim 3, (b) determining the amount of non-IgE antibody bound to the peptide, thereby determining the amount of non-IgE antibody specific to an epitope of a protein in the sample.

10. The method of claim 9 where the non-IgE antibodies are IgA, and/or IgG, and/or IgM.

11. The method of claim 10 where the majority of non-IgE antibody levels are determined by measuring epitope-specific kappa-chain plus lambda-chain antibodies.

12. A method of diagnosing an immune disorder comprising performance of matched, epitope-specific immunoassays of claims 8 and 9 in parallel and: (a) dividing the epitope-specific IgE level by the matching specific kappa+lambda antibody level; (b) multiplying the quotient value by 1,000,000 to derive a relative quotient value; (c) assigning as positive, relative quotient values that are equal to or greater than 0.5; and
(d) inspecting the individual test results of the dimer points listed in claim 5 for disease-positive dimeric matches.

13. The method of claim 12, wherein the immune disorder is multiple sclerosis.

14. A method of treating multiple sclerosis or a multiple sclerosis-like condition comprising administering a composition of claim 3 to the subject in a therapeutically effective amount and manner sufficient to neutralize the specific IgE autoantibody and alleviate at least one symptom and/or physical finding of multiple sclerosis.

15. The method of claim 14 wherein only one of the dimeric IgE attachments is blocked thus preventing IgE dimers from forming and thereby abrogating or diminishing mast cell degranulation and disease onset and/or continuation.

16. The method of claims 14 and 15 where individual constructs listed in claim 3 are administered alone or in combination to treat multiple sclerosis.

17. A method for assessing positive therapeutic efficacy following application of the methods of claims 14, 15, and 16 by applying the diagnostic method of claim 12 and attaining quotient values that are zero or approaching zero.
